SEQUENCE LISTING (7 sequences)

SEQ ID NO: 1 - DNA, 30 bp, Artificial sequence
  synthetic oligonucleotide
ttctttgtgatgtgtactttcatctcacag

SEQ ID NO: 2 - DNA, 25 bp, Artificial sequence
  Synthetic oligonucleotide
tgtgatgtgtactttcatctcacag

SEQ ID NO: 3 - DNA, 25 bp, Artificial sequence
  Synthetic oligonucleotide
ttgtgatgtgtactttcatctcaca

SEQ ID NO: 4 - DNA, 25 bp, Artificial sequence
  Synthetic oligonucleotide
tttgtgatgtgtactttcatctcac

SEQ ID NO: 5 - DNA, 25 bp, Artificial sequence
  Synthetic oligonucleotide
ctttgtgatgtgtactttcatctca

SEQ ID NO: 6 - DNA, 25 bp, Artificial sequence
  Synthetic oligonucleotide
tctttgtgatgtgtactttcatctc

SEQ ID NO: 7 - DNA, 25 bp, Artificial sequence
  Synthetic oligonucleotide
ttctttgtgatgtgtactttcatct

(19) United States
(12) Patent Application Publication
(10) Pub. No.: US 2006/0292585 A1
(43) Pub. Date: Dec. 28, 2006

(54) Analysis of methylation using nucleic acid arrays

(75) Inventors: Shivani Nautiyal, San Francisco, CA (US); John E. Blume, Danville, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/213,273

(22) Filed: Aug. 26, 2005

Related U.S. Application Data
(60) Provisional application No. 60/694,103, filed on Jun. 24, 2005.

(51) Int. Cl. C12Q 1/68; C12P 19/34

(52) U.S. Cl. 435/6; 435/91.2

(57) ABSTRACT

Methods of analyzing DNA to determine the methylation status of a plurality of cytosines are disclosed. In one aspect genomic DNA is fragmented, fragments are circularized, the circles are treated with a methylation sensitive enzyme to enrich for circles with methylated sites or with a methylation dependent enzyme to enrich for circles with unmethylated sites, and the circles are amplified. The amplified product is fragmented, labeled and hybridized to an array of probes. The array of probes may be a tiling array or an array of junction probes. The hybridization pattern is analyzed to determine methylation status of cytosines.

### RELATED APPLICATIONS

This application claims the priority of U.S. Provisional Application No. 60/694,103 filed Jun. 24, 2005, the entire disclosure of which is incorporated herein by reference in its entirety.

### FIELD OF THE INVENTION

The present invention relates to methods for detecting methylation using arrays of nucleic acids.

### BACKGROUND OF THE INVENTION

The genomes of higher eukaryotes contain the modified nucleoside 5-methyl cytosine (5-meC). This modification is usually found as part of the dinucleotide CpG. Cytosine is converted to 5-methylcytosine in a reaction that involves flipping a target cytosine out of an intact double helix and transfer of a methyl group from S-adenosylmethionine by a methyltransferase enzyme (Klimasauskas et al., Cell 76:357-369, 1994). This enzymatic conversion is the only epigenetic modification of DNA known to exist in vertebrates and is essential for normal embryonic development (Bird, Cell 70:5-8, 1992; Laird and Jaenisch, Human Mol. Genet. 3:1487-1495, 1994; and Li et al., Cell 69:915-926, 1992).

The frequency of the CpG dinucleotide in the human genome is only about 20% of the statistically expected frequency, possibly because of spontaneous deamination of 5-meC to T (Schoreret et al., Proc. Natl. Acad. Sci. USA 89:957-961, 1992). There are about 28 million CpG doublets in a haploid copy of the human genome and it is estimated that about 70-80% of the cytosines at CpGs are methylated. Regions where CpG is present at levels that are approximately the expected frequency are referred to as “CpG islands” (Bird, A. P., Nature 321:209-213, 1986). These regions have been estimated to comprise about 1% of vertebrate genomes and account for about 15% of the total number of CpG dinucleotides. CpG islands are typically between 0.2 and 1 kb in length and are often located upstream of housekeeping and tissue-specific genes. CpG islands are often located upstream of transcribed regions, but may also extend into transcribed regions. About 2-4% of cytosines are methylated and probably the majority of cytosines that are 5′ of Gs are methylated. Most of the randomly distributed CpGs are methylated, but only about 20% of the CpGs in CpG islands are methylated.

DNA methylation is an epigenetic determinant of gene expression. Patterns of CpG methylation are heritable, tissue specific, and correlate with gene expression. The consequence of methylation is usually gene silencing. DNA methylation also correlates with other cellular processes including embryonic development, chromatin structure, genomic imprinting, somatic X-chromosome inactivation in females, inhibition of transcription and transposition of foreign DNA and timing of DNA replication. When a gene is highly methylated it is less likely to be expressed, possibly because CpG methylation prevents transcription factors from recognizing their cognate binding sites. Proteins that bind methylated DNA may also recruit histone deacetylase to condense adjacent chromatin. Such “closed” chromatin structures prevent binding of transcription factors. Thus the identification of sites in the genome containing 5-meC is important in understanding cell-type specific programs of gene expression and how gene expression profiles are altered during both normal development and diseases such as cancer. Precise mapping of DNA methylation patterns in CpG islands has become essential for understanding diverse biological processes such as the regulation of imprinted genes, X chromosome inactivation, and tumor suppressor gene silencing in human cancer caused by increase methylation.

Methylation of cytosine residues in DNA plays an important role in gene regulation. Methylation of cytosine may lead to decreased gene expression by, for example, disruption of local chromatin structure, inhibition of transcription factor-DNA binding, or by recruitment of proteins which interact specifically with methylated sequences and prevent transcription factor binding. DNA methylation is required for normal embryonic development and changes in methylation are often associated with disease. Genomic imprinting, X chromosome inactivation, chromatin modification, and silencing of endogenous retroviruses all depend on establishing and maintaining proper methylation patterns. Abnormal methylation is a hallmark of cancer cells and silencing of tumor suppressor genes is thought to contribute to carcinogenesis. Methylation mapping using microarray-based approaches may be used, for example, to profile cancer cells revealing a pattern of DNA methylation that may be used, for example, to diagnose a malignancy, predict treatment outcome or monitor progression of disease. Methylation in eukaryotes can also function to inhibit the activity of viruses and transposons, see Jones et al., EMBO J. 17:6385-6393 (1998). Alterations in the normal methylation process have also been shown to be associated with genomic instability (Lengauer et al., Proc. Natl. Acad. Sci. USA 94:2545-2550, 1997). Such abnormal epigenetic changes may be found in many types of cancer and can serve as potential markers for oncogenic transformation.

### SUMMARY OF THE INVENTION

Methods for analyzing the methylation status of cytosines in genomic DNA are disclosed. In many embodiments the genomic DNA sample is fragmented and the fragments are circularized. Enzymes with differential sensitivity to methylation of the enzyme recognition site are used to cleave circles after fragmentation or to cleave linear fragments before circularization. In some aspects linear fragments are digested, for example, using an exonuclease, leaving circular fragments intact. The remaining circular fragments may then be amplified. The amplified fragments can be characterized by hybridization to an array of probes. Hybridization patterns are analyzed to determine methylation patterns.

In one aspect the sample is fragmented with a first restriction enzyme that is insensitive to the methylation status of cytosines and a second restriction enzyme that is sensitive to the methylation status of cytosines. The ends of the resulting fragments are ligated together to form circles. Fragments that are not circularized may be removed by digestion with an exonuclease. The circles are amplified and the amplification product is fragmented, labeled and hybridized to an array of probes that includes probes that recognize the novel junction sequences formed by circularization. The hybridization pattern is analyzed to determine the presence of selected junctions, where the presence of selected junctions indicates the methylation state of selected cytosines.

Junctions can be predicted by computer implemented modeling of digestion. The junction probes can also be designed by computer methods. Different fragments result depending on whether a site is methylated and different junctions result. Predicted junctions may be identified using a computer system and the oligonucleotide probes are designed to detect the presence of a plurality of the predicted junctions. A database of methylation informative junction sequences may be obtained by computer modeling of fragmentation and ligation using methylation sensitive and insensitive restriction enzymes. A computer may be used to analyze hybridization pattern to compare the expected hybridization results and make calls about whether individual restriction sites were methylated or not. The array preferably interrogates more than 1,000, more than 10,000 or more than 100,000 different possible sites of methylation. The array may be a single solid support with a plurality of different probes arranged in features of known location on the array or a plurality of solid supports with one or more probes attached to each support. The support may be, for example, a chip, a glass slide or a bead.

In some aspects the first enzyme is BsaW I, BsoB I, BssS I, Msp I or Taq I and the second enzyme is Aat II, Aci I, Acl I, Afe I, Age I, Asc I, Ava I, BmgB I, BsaA I, BsaH I, BspD I, Eag I, Fse I, Fau I, Hpa II, HinP1 I, Nar I, Hin6I, HapII or SnaB I. Preferably the first and second enzymes generate compatible cohesive ends. Alternatively the ends may be end filled to generate blunt ends and the blunt ends may be ligated together.

In some aspects the hybridization pattern obtained from the sample is compared to a control hybridization pattern. The control hybridization pattern in some aspects is obtained by treating an aliquot of the sample with the first restriction enzyme but not the second restriction enzyme. As above, the fragments are ligated to form circles and the circles, linear fragments are digested with exonuclease and the circles are amplified. The amplification product is fragmented, labeled and hybridized to an array of the same design as the sample was hybridized to. The control hybridization pattern that is generated can be compared to the hybridization pattern obtained for the sample to identify differences, the differences are indicative of methylation.

In some aspects the hybridization pattern of a sample is compared to a hybridization pattern from a control sample, where the control sample is fragmented with the same first restriction enzyme as the experimental sample and with a methylation insensitive isoschizomers of the second restriction enzyme used to fragment the experimental sample. The fragments are ligated end-to-end to form circles and can then be digested with an exonuclease to remove linear fragments. The circles are amplified and the amplification product is fragmented, labeled and hybridized to an array to generate a control hybridization pattern to be compared with the hybridization pattern of the sample. The control sample may be an aliquot of the experimental sample.

In some aspects the methods are used to classify a tissue into a class, for example, a known tumor class. The hybridization pattern obtained from the tissue sample, using the disclosed methods, is compared to hybridization patterns from samples from tissues of known tumor class, obtained using the disclosed methods.

In one aspect arrays that may be used in connection with the disclosed methods are also disclosed. In some aspects arrays comprising probes to junctions formed by ligated fragments end-to-end are disclosed.

In another aspect the probes are complementary to restriction fragments resulting from digestion with a first enzyme that also include a restriction site for a second enzyme that is methylation sensitive or methylation dependent. Probe sets may be directed at any region within the fragment and do not need to hybridize to the restriction enzyme recognition site. In some aspects the probes of the array may be allele specific and may be perfectly complementary to one allele of a polymorphism within the fragment. This may be used to detect allele specific methylation. The array preferably includes more than 100,000 probes and more than 90% of the probes are preferably experimental probes. The array preferably includes control probes as well. In a preferred aspect the probes of the array are perfectly complementary to regions in the human, mouse or rat genome. The array may include probes to a plurality of different species.

In another aspect a genomic DNA sample is digested with a methylation sensitive restriction enzyme, the fragments are ligated end-to-end to form circles and the circles are digested with a methylation dependent enzyme, for example, McrBC. The sample is then treated with an exonuclease to remove linear fragments and the circles are amplified. The amplification products are fragmented, labeled and hybridized to an array. The hybridization pattern is analyzed. Fragments that result from the digestion with the methylation sensitive enzyme and contain a recognition site for the MD enzyme were not methylated at the recognition site for the MD enzyme.

In another aspect a sample is enriched for methylated regions by fragmenting the sample, ligated the sample fragments end-to-end to form circles, mixing the circles with a methylation sensitive enzyme and an exonuclease.

In another aspect a sample is enriched for unmethylated regions by fragmenting with an enzyme that is methylation sensitive, ligating the resulting fragments end-to-end to form circles and treating the samples with a methylation dependent restriction enzyme, such as McrBC. Fragments with a methylated McrBC site will be linearlized and can then be digested with exonuclease, such as Lambda exonuclease, with or without Exonuclease I.

In another aspect the sample is fragmented with a restriction enzyme that is methylation insensitive and an enzyme that is methylation sensitive. The fragments are ligated to form circles. A plurality of molecular inversion probes are hybridized to the circles. The MIPs are designed so that they detect the presence of selected junctions so only when a junction is present will the ends of the MIP be juxtaposed and available for ligation. After ligation, uncircularized MIPs are digested with exonuclease and the remaining MIPs are amplified and detected. The MIP preferably includes a tag sequence and universal priming sites for PCR amplification.

### BRIEF DESCRIPTION OF THE DRAWINGS

FIG. 1. shows a schematic of one embodiment. The DNA is digested with a methylation insensitive enzyme, circularized and either treated with methylation sensitive or methylation dependent enzymes and products detected.

FIG. 2 shows a schematic of a method that includes digestion with a methylation sensitive enzyme, circularization of fragments, digestion with a methylation dependent enzyme, digestion with exonuclease, amplification by RCA, and detection on an array.

FIG. 3 shows a schematic of a method that includes digestion with a methylation insensitive enzyme, TaqI, and digestion with a methylation sensitive enzyme HpaII. The enzymes generate compatible overhangs. Fragments are circularized and circles are amplified. The amplified circles are analyzed by hybridization to an array of junction probes that are complementary to the different predicted junctions.

FIG. 4 shows a schematic of a method of identifying methylated sites by comparing hybridization patterns of a sample fragmented with isoschizomers with differential methylation sensitivity.

FIG. 5 shows a schematic of a method of detection of junctions using molecular inversion probes.

FIG. 6 shows a schematic of a method that includes fragmentation and adaptor ligation of a nucleic acid sample, followed by bisulfite treatment of the adaptor ligated fragments. The fragments are then amplified by PCR, resulting in size selection and complexity reduction and hybridized to an array.

FIG. 7 illustrates an example of a computer system that may be utilized to execute the software of an embodiment of the invention.

FIG. 8 illustrates a system block diagram of the computer system of FIG. 7.

### DETAILED DESCRIPTION OF THE INVENTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being, but may also include other organisms including but not limited to mammals, plants, fungi, bacteria or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with hybridization to an array, the sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675. The sample may be amplified on the array. See, for example, U.S. Pat. No. 6,300,070 which is incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245), rolling circle amplification (RCA) (for example, Fire and Xu, PNAS 92:4641 (1995) and Liu et al., J. Am. Chem. Soc. 118:1587 (1996)) and nucleic acid based sequence amplification (NABSA), (See, U.S. Pat. Nos. 5,409,818, 5,554,517, and 6,063,603). Other amplification methods that may be used are described in, U.S. Pat. Nos. 5,242,794, 5,494,810, 4,988,617 and in U.S. Ser. No. 09/854,317. Other amplification methods are also disclosed in Dahl et al., Nuc. Acids Res. 33(8):e71 (2005) and circle to circle amplification (C2CA) Dahl et al., PNAS 101:4548 (2004). Locus specific amplification and representative genome amplification methods may also be used.

Additional methods of sample preparation and techniques for reducing the complexity of a nucleic sample are described in Dong et al., Genome Research 11, 1418 (2001), in U.S. Pat. Nos. 6,872,529, 6,361,947, 6,391,592 and 6,107,023, U.S. Patent Publication Nos. 20030096235 and 20030082543 and U.S. patent application Ser. No. 09/916,135.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO 99/47964), each of which also is hereby incorporated by reference in its entirety for all purposes. Instruments and software may also be purchased commercially from various sources, including Affymetrix.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

Methods for detection of methylation status are disclosed, for example, in Fraga and Esteller, BioTechniques 33:632-649 (2002) and Dahl and Guldberg Biogerontology 4:233-250 (2003). Methylation detection using bisulfite modification and target specific PCR have been disclosed, for example, in U.S. Pat. Nos. 5,786,146, 6,200,756, 6,143,504, 6,265,171, 6,251,594, 6,331,393, and 6,596,493. U.S. Pat. No. 6,884,586 disclosed methods for methylation analysis using nicking agents and isothermal amplification.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), 10/065,856, 10/065,868, 10/328,818, 10/328,872, 10/423,403, and 60/482,389.

All documents, i.e., publications and patent applications, cited in this disclosure, including the foregoing, are incorporated by reference herein in their entireties for all purposes to the same extent as if each of the individual documents were specifically and individually indicated to be so incorporated by reference herein in its entirety.

b) Definitions

“Adaptor sequences” or “adaptors” are generally oligonucleotides of at least 5, 10, or 15 bases and preferably no more than 50 or 60 bases in length; however, they may be even longer, up to 100 or 200 bases. Adaptor sequences may be synthesized using any methods known to those of skill in the art. For the purposes of this invention they may, as options, comprise primer binding sites, recognition sites for endonucleases, common sequences and promoters. The adaptor may be entirely or substantially double stranded or entirely single stranded. A double stranded adaptor may comprise two oligonucleotides that are at least partially complementary. The adaptor may be phosphorylated or unphosphorylated on one or both strands.

Adaptors may be more efficiently ligated to fragments if they comprise a substantially double stranded region and a short single stranded region which is complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible. Blunt ends can be converted to sticky ends using the exonuclease activity of the Klenow fragment. For example when DNA is digested with PvuII the blunt ends can be converted to a two base pair overhang by incubating the fragments with Klenow in the presence of dTTP and dCTP. Overhangs may also be converted to blunt ends by filling in an overhang or removing an overhang.

Methods of ligation will be known to those of skill in the art and are described, for example in Sambrook et at. (2001) and the New England BioLabs catalog both of which are incorporated herein by reference for all purposes. Methods include using T4 DNA Ligase which catalyzes the formation of a phosphodiester bond between juxtaposed 5′ phosphate and 3′ hydroxyl termini in duplex DNA or RNA with blunt and sticky ends; Taq DNA Ligase which catalyzes the formation of a phosphodiester bond between juxtaposed 5′ phosphate and 3′ hydroxyl termini of two adjacent oligonucleotides which are hybridized to a complementary target DNA; E. coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′->5′ phosphodiester bond, substrates include single-stranded RNA and DNA as well as dinucleoside pyrophosphates; or any other methods described in the art. Fragmented DNA may be treated with one or more enzymes, for example, an endonuclease, prior to ligation of adaptors to one or both ends to facilitate ligation by generating ends that are compatible with ligation.

Adaptors may also incorporate modified nucleotides that modify the properties of the adaptor sequence. For example, phosphorothioate groups may be incorporated in one of the adaptor strands. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified backbone of an S-Oligo is resistant to the action of most exonucleases and endonucleases. Phosphorothioates may be incorporated between all residues of an adaptor strand, or at specified locations within a sequence. A useful option is to sulfurize only the last few residues at each end of the oligo. This results in an oligo that is resistant to exonucleases, but has a natural DNA center.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “array plate” as used herein refers to a body having a plurality of arrays in which each microarray is separated by a physical barrier resistant to the passage of liquids and forming an area or space, referred to as a well, capable of containing liquids in contact with the probe array.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa, Nucleic Acids Res. 12:203 (1984), incorporated herein by reference.

The term “epigenetic” as used herein refers to factors other than the primary sequence of the genome that affect the development or function of an organism, they can affect the phenotype of an organism without changing the genotype. Epigenetic factors include modifications in gene expression that are controlled by heritable but potentially reversible changes in DNA methylation and chromatin structure. Methylation patterns are known to correlate with gene expression and in general highly methylated sequences are poorly expressed.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004, available at Affymetrix.com.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” as used herein refers to an intentionally created collection of nucleic acids which can be prepared either synthetically or biosynthetically and screened for biological activity in a variety of different formats (for example, libraries of soluble molecules; and libraries of oligos tethered to beads, chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

Restriction enzymes or restriction endonucleases and their properties are well known in the art. A wide variety of restriction enzymes are commercially available, from, for example, New England Biolabs. Restriction enzymes recognize a sequence specific sites (recognition site) in DNA. Typically the recognition site varies from enzyme to enzyme and may also vary in length. Isoschizomers are enzymes that share the same recognition site. Restriction enzymes may cleave close to or within their recognition site or outside of the recognition site. Often the recognition site is symmetric because the enzyme binds the double stranded DNA as homodimers. Recognition sequences may be continuous or may be discontinuous, for example, two half sites separated by a variable region. Cleavage can generate blunt ends or short single stranded overhangs.

In preferred aspects of the present invention enzymes that include at least one CpG dinucleotide in the recognition site may be used. Enzymes with a recognition site that includes the sequence CCGG include, for example, Msp I, Hpa II, Age I, Xma I, Sma I, NgoM IV, Nae I, and BspE I. Enzymes with a recognition site that includes the sequence CGCG include, for example, BstU I, Mlu I, Sac II, BssH II and Nru I. Enzymes with a recognition site that includes the sequence GCGC include, for example, Hin P1 I, Hha I, Afe I, Kas I, Nar I, Sfo I, Bbe I, and Fsp I. Enzymes with a recognition site that includes the sequence TCGA include, for example, Taq I, Cla I, BspD I, PaeR7 I, Tli I, Xho I, Sal I, and BstB I. For additional enzymes that contain CpG in the recognition sequence. See, for example, the New England Biolabs catalog and web site. In some aspects two restriction enzymes may have a different recognition sequence but generate identical overhangs or compatible cohesive ends. For example, the overhangs generated by cleavage with Hpa II or Msp I can be ligated to the overhang generated by cleavage with Taq I. Some restriction enzymes that include CpG in the recognition site are unable to cleave if the site is methylated, these are methylation sensitive. Other enzymes that contain CpG in their recognition site can cleave regardless of the presence of methylation, these are methylation insensitive. Examples of methylation insensitive enzymes, that include a CpG in the recognition site, include BsaW I (WCCGGW), BsoB I, BssS I, Msp I, and Taq I. Examples of methylation sensitive enzymes, that include a CpG in the recognition site, include Aat II, Aci I, Acl I, Afe I, Age I, Asc I, Ava I, BmgB I, BsaA I, BsaH I, BspD I, Eag I, Fse I, Fau I, Hpa II, HinP1 I, Nar I, and SnaB I.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

The term “wafer” as used herein refers to a substrate having surface to which a plurality of arrays are bound. In a preferred embodiment, the arrays are synthesized on the surface of the substrate to create multiple arrays that are physically separate. In one preferred embodiment of a wafer, the arrays are physically separated by a distance of at least about 0.1, 0.25, 0.5, 1 or 1.5 millimeters. The arrays that are on the wafer may be identical, each one may be different, or there may be some combination thereof. Particularly preferred wafers are about 8″×8″ and are made using the photolithographic process.

Methylation Analysis

Mammalian methylation patterns are complex and change during development, see van Steensel and Henikoff BioTechniques 35: 346-357 (2003). Methylation in promoter regions is generally accompanied by gene silencing and loss of methylation or loss of the proteins that bind to the methylated CpG can lead to diseases in humans, for example, Immunodeficiency Craniofacial Syndrome and Rett Syndrome, Bestor (2000) Hum. Mol. Genet. 9:2395-2402. DNA methylation may be gene-specific and occurs genome-wide.

Methods for detecting methylation status have been described in, for example U.S. Pat. Nos. 6,214,556, 5,786,146, 6,017,704, 6,265,171, 6,200,756, 6,251,594, 5,912,147, 6,331,393, 6,605,432, and 6,300,071 and US Patent Application publication Nos. 20030148327, 20030148326, 20030143606, 20030082609 and 20050009059, each of which are incorporated herein by reference. Other array based methods of methylation analysis are disclosed in U.S. patent application Ser. No. 11/058,566. For a review of some methylation detection methods, see, Oakeley, E. J., Pharmacology & Therapeutics 84:389-400 (1999). Available methods include, but are not limited to: reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), sodium bisulfite, combined bisulphate-restriction analysis, and methylation sensitive single nucleotide primer extension.

In one aspect the methods of the invention relate to methods that result in enrichment and amplification of methylated or unmethylated sequences from a sample. The amplified sample may then be interrogated by hybridization to a high density array of oligonucleotide probes to determine if a plurality of selected sequences of interest are present or absent in either sample. Separate samples may be enriched for methylated sequences or enriched for unmethylated sequences, depending on treatment. The methylation state of a plurality of sequences can be determined using the methods. Preferably more than 1,000, 5,000, 10,000, or more than 100,000 different cytosines are analyzed for methylation in parallel. The methods may be used to identify biomarkers of epigenetic regulation based on methylation of surrounding CpGs.

In many aspects the methods include digestion with enzymes that have different sensitivities to methylation, circularization of individual fragments by ligation and amplification of the circular fragments. Circularization generates junctions that are not naturally found in the genome. Detection of the junctions and analysis of which junctions are present may be used to determine if selected sites were methylated.

In one aspect genomic DNA is fragmented with an enzyme that is methylation insensitive, for example, the enzyme recognition site may not include a CpG or the site may include a CpG and cleavage is insensitive to methylation. The resulting fragments are circularized by ligation and the circles are digested with an enzyme that is methylation sensitive and cleaves preferentially at the corresponding recognition site if the site is unmethylated. Circular fragments that contain an unmethlyated recognition site for the methylation sensitive enzyme will be linearized, while circular fragments that contain only methylated recognition sites for the methylation sensitive enzyme will remain circular and can be detected. The products of this fragmentation may optionally be subjected to digestion with an exonuclease to remove linear fragments. The remaining circular fragments (containing only methylated recognition sites for the methylation sensitive enzyme or no sites) may then be detected. The circular fragments or portions thereof may also be amplified, for example by a RCA method. The amplification product may be fragmented and the fragments may be labeled, for example, by end labeling using TdT and incorporating a biotin labeled nucleotide. The labeled fragments may be hybridized to an array of probes and the pattern of hybridization can be analyzed to determine methylation. Hybridization at probes that are complementary to a predicted circle that contains a recognition site for a methylation sensitive enzyme may indicate that the restriction site was methylated. The probe may be to any region of the circle.

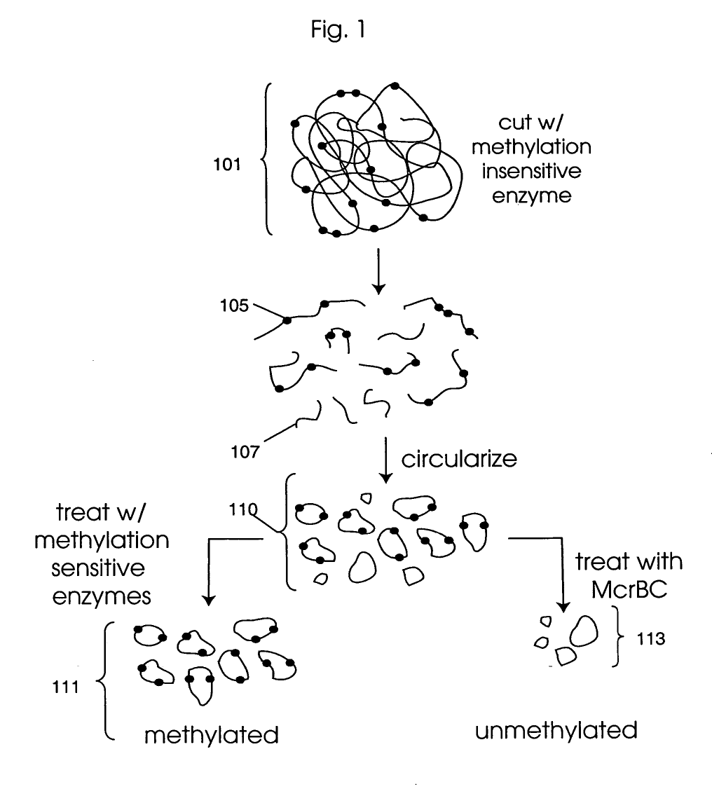

In one aspect (FIG. 1) a sample [101] is fragmented by a method that is insensitive to methylation state. Some of the fragments [105] contain methylated sites (indicated by small circles) and others [107] do not. The fragments are circularized, some of the circles contain methylated sites and others do not. An aliquot of the sample is treated with enzymes that are methylation sensitive to generate a product that is enriched for methylated circles [111]. A second aliquot is treated with a methylation dependent enzyme so to generate a product that is enriched for unmethylated circles [113].

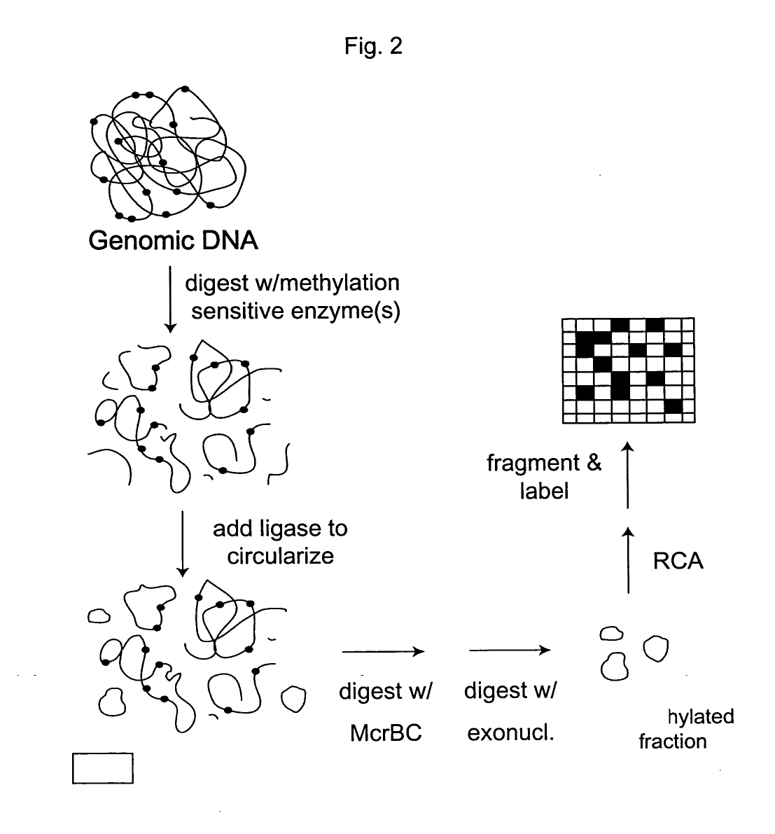

In another aspect (FIG. 2) genomic DNA with methylated cytosines is fragmented with a methylation sensitive enzyme that cleaves wherever its recognition site is unmethylated. The fragments are then circularized by ligation and digested with an enzyme that cleaves only when its recognition site is methylated. The sample is then digested with an exonuclease to digest linear DNA. The remaining circles, which are enriched for unmethylated DNA, can be amplified by an amplification method that preferentially amplifies circular nucleic acids, for example, RCA. The resulting fragments may be labeled and hybridized to an array to detect regions that are present in the unmethylated fraction.

In contrast to methods where DNA is randomly cleaved, the use of restriction enzymes provide a predictable pattern of cleavage which allows prediction of ligation junctions so that probes may be designed for the junctions formed by self-ligation of the opposite ends of a fragment. The junctions are expected to represent novel sequences that would not otherwise be present and can be interrogated. Junctions include a first half that is from the first end of the fragment and a second half that is from the second end of the fragment. Circles may also form between multiple fragments and the junctions may include a portion from two or more fragments. The junction probes may be probes that are attached to a solid support and are complementary to the junction, typically including a portion that is complementary to a region in the first half of the junction and a region that is complementary to a region in the second half of the junction. A probe set that is tiled across a junction may be used.

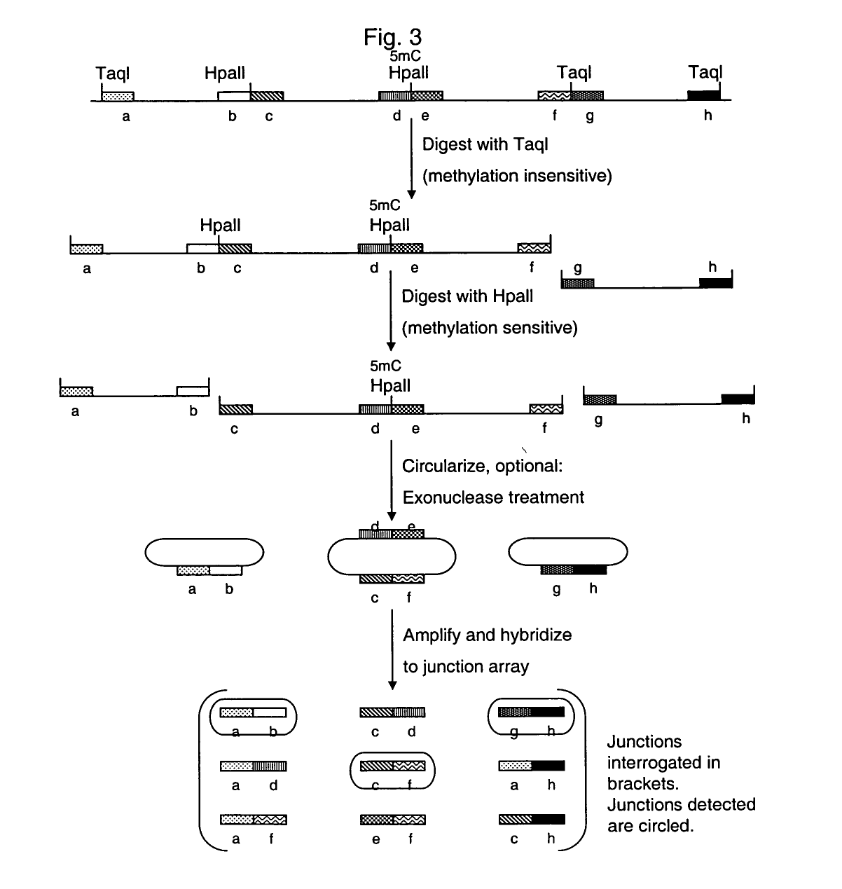

In another embodiment (FIG. 3) an enzyme based scheme is used for determining site-specific methylation. The genomic DNA is digested with at least one enzyme that is methylation insensitive (MI), and at least one enzyme that is methylation sensitive (MS). The digests may be performed sequentially or simultaneously. Fragments that are cleaved by the MI enzyme that contain an unmethylated recognition site for the MS are cleaved into sub-fragments. Fragments that contain a methylated recognition site for the MS are not cleaved into sub-fragments. Following ligation, the fragments that are not cleaved, may be ligated to form a junction between the sequence immediately downstream of one MI site and the sequence immediately upstream of a second MI site, this circular fragment may contain an undigested MS site.

Preferably, the ligation conditions are designed to favor ligation between the two ends of the same fragment and to select against ligation between the end of a first fragment and the end of a second fragment. Preferably the majority, more than 50, 60, 70 80 or 90% of the circles will be formed by a single fragment. Reducing the concentration of fragments favors formation of circles by a single fragment. If the MS site is not-methylated the fragment may be cleaved at the MS site and upon ligation a junction will form between the sequence immediately downstream of an MI site and immediately upstream of an MS site or the sequence immediately 5′ of an MI site and immediately 3′ of an MS site. The junctions are novel sequences that were not present in the genome and probes to detect each of these possible outcomes are preferably designed and synthesized on an array. The presence or absence of hybridization signal above background is indicative of the presence or absence of a junction. For example, in FIG. 3 half junctions (a) through (h) are shown. The (a), (f), (g) and (h) half junctions correspond to regions immediately upstream, (f) and (h), or downstream, (a) and (g), of a MI site. After cleavage with the MI enzyme the MI half junctions that are generated are (a), (f) and (g), (h), with each defining a different fragment. The (b), (c), (d), and (e), half junctions are immediately up or down stream of a MS site. In the figure, the MS site flanked by (b) and (c) is not methylated and the MS site flanked by (d), (e) is methylated. After fragmentation with the MS enzyme the unmethylated site will be digested to generate sub-fragments flanked by (a) and (b), (c) and (f); the fragment flanked by (g) and (h) that was generated by cleavage with the MI enzyme remains in tact. The methylated site will not be fragmented leaving a fragment flanked by (c) and (f) and containing (d) and (e). The array can contain probes that detect specific junctions as shown. The hybridization pattern can be analyzed to determine the methylation state of selected MS sites. For example, if the probe set to the junction formed by (a) and (b) is present this is indicative that the first MS site was cleaved and that the site was unmethylated. The presence of the junction formed by (c) and (f), also the absence of the junction formed by (c) and (d) and (e) and (f), indicates that the MS site flanked by (d) and (e) was not cleaved.

The presence of junctions that include a region upstream of a MS site or downstream of a MS site indicates that the MS site was not methylated and was cleaved by the MS enzyme. The presence of a junction formed by the joining of a downstream half and an upstream half of sites (MS or MI) that flank an MS site is an indication that the MS site was methylated and not digested by the MS enzyme. For example, detection of the junction formed by the joining of (c) and (f) in FIG. 3 indicates that the HpaII site flanked by (d) and (e) was methylated. Also, the absence of junctions that include (d) and (e) indicates methylation of that site.

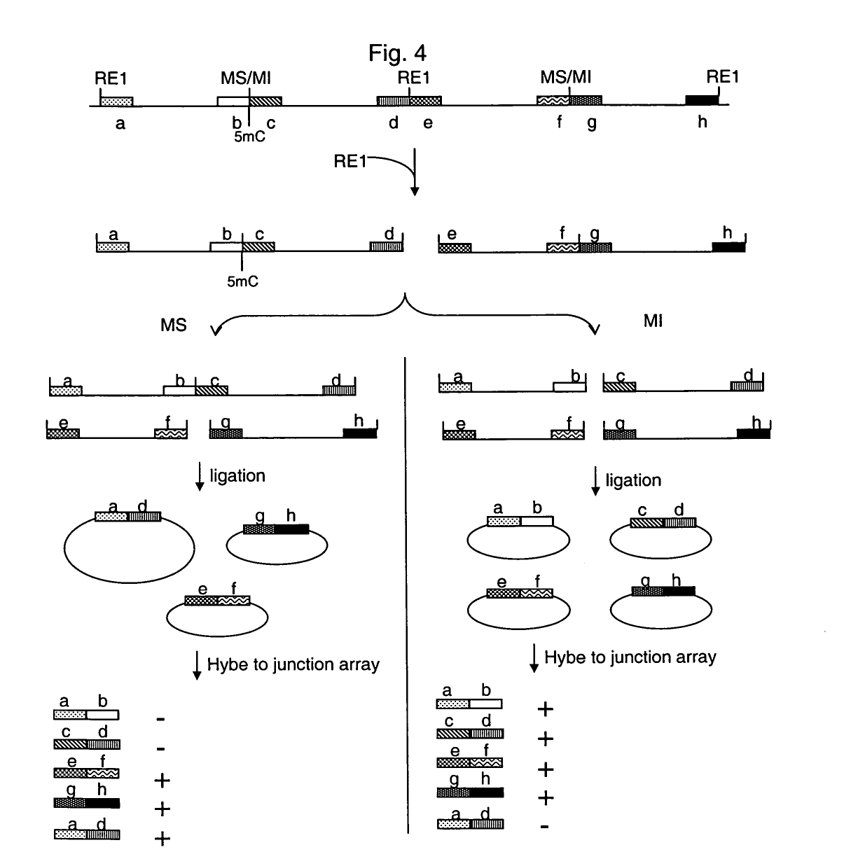

In some aspects the hybridization pattern resulting from treatment of a sample with a methylation sensitive enzyme is compared to a hybridization pattern resulting from treatment of an aliquot of the sample or a control sample with a methylation insensitive isoschizomer of the methylation sensitive enzyme as shown in FIG. 4. The sample treated with the methylation insensitive enzyme provides a positive control for the sample treated with the methylation sensitive enzyme.

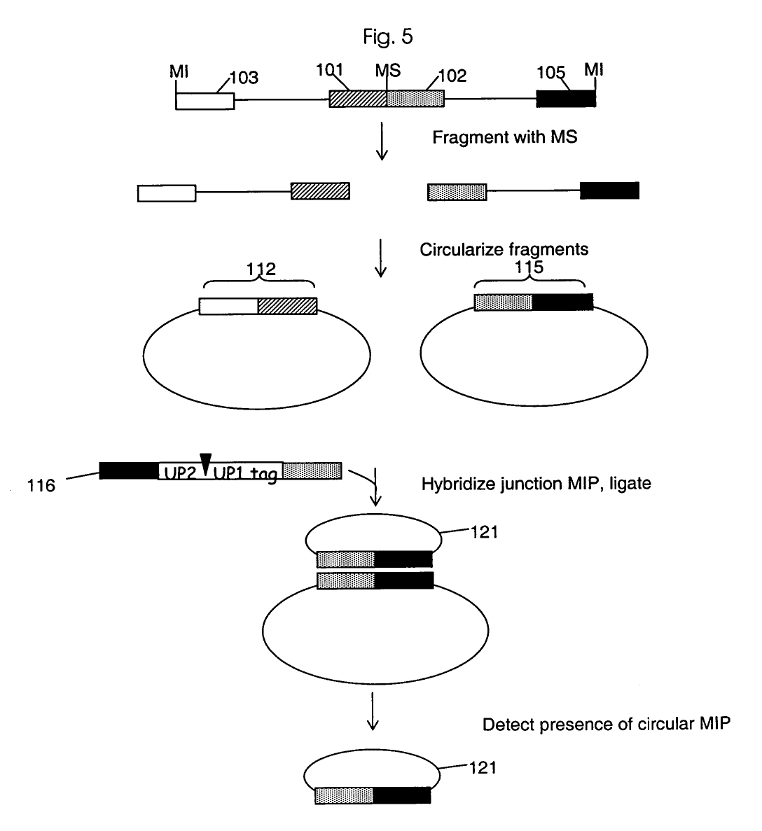

Junctions may be detected by hybridization to junction specific probes where the probes are attached to one or more solid supports. In another aspect (FIG. 5) junctions are detected using a molecular inversion probe (MIP) based assay, described in Hardenbol et al., Genome Res. 15:269-275 (2005) and in U.S. Pat. No. 6,858,412. The double stranded genomic DNA to be interrogated for methylation at a methylation sensitive restriction site (MS) is fragmented with an MI enzyme and the MS enzyme. The sequence upstream [101] or downstream [102] of the MS site will form part of a junction with upstream [103] or downstream [105] sequence if cleavage occurs at MS. Presence of the junctions [112 and 115] indicates that the MS site was not methylated. The MIP [116] has ends that are complementary to junction halves [102 and 105] so that only when the junction [115] forms can the MIP hybridize to the circularized fragment in a way that juxtaposes the ends of the MIP for ligation to generate a circularized MIP [121]. The MIP also includes a tag sequence, sites for internal cleavage and sites for universal primer binding (UP1 and UP2) to facilitate amplification and detection by PCR as described in Hardenbol et al. The presence of absence of selected MIP probes that correspond to known or predicted junctions and contain known tag sequence may be detected using an array of tag probes that are complementary to the tags in the MIPs. The same array may be used to detect methylation at many different cytosines by designing different sets of MIPs. In some aspects sets of more than 1,000, 5,000, 10,000 or 100,000 different MIPs are designed, each targeting a different predicted junction and each having a different tag sequence.

An optional exonuclease treatment may be included after circularization to remove uncircularized fragments. Methods of self-ligation of the ends of restriction fragments followed by rolling circle amplification have been disclosed, for example, in Wang et al., Gen. Res. 14:2357-2366, 2004, which also discloses uses for the amplified material and methods of analysis of the amplified material and is incorporated herein in its entirety for all purposes. The circularized fragments may be amplified using an amplification method that preferentially amplifies circles over non-circles (linear fragments), for example, RCA or C2CA (see, Dahl et al., PNAS 101:4548 (2004)). The amplification product may be fragmented, labeled and hybridized to probe arrays. The probe arrays may be, for example, tiling arrays or arrays of junction probes. Tiling arrays are arrays where the probes are spaced at defined equal intervals over a genome or genomic region, minus repetitive elements. For example, probes may be spaced so that they are separated by a specified number of bases, for example, 5, 10, 25, 35 or 100 base pair spacing between probe ends or between probe centers. The probes of tiling arrays may also overlap, for example the spacing may be 1 or more base pairs between probe centers. For more information about tiling arrays, methods for using tiling arrays and analysis of tiling array data see, for example, Kapranov et al., Genome Res. 15:987-997 (2005) and Cheng et al., Science 308:1149-1154 (2005). For an array of junction probes, the junction probes may be designed to detect predicted junctions that are present or absent depending on the methylation state of the cytosines in the recognition site for the enzyme. If a site is methylated the site will not be cleaved and probes to the regions flanking that site will not detect signal above background levels. Junction probes may be designed to interrogate a subset of possible junctions. A database of possible junctions formed by circularization of restriction fragments may be identified by computer modeling of digestion and ligation reactions and used to design the array and to analyze the hybridization pattern of a sample to the array. The computer may also be used to select junctions for interrogation based on selection criteria. The criteria may include, for example, selection of junctions formed by ligation of fragments that include an internal restriction site for a selected enzyme, exclusion of junction sequences that cross hybridize with other sequences in the genome, and exclusion of junction sequences that have internal secondary structure.

In a first aspect genomic DNA is fragmented using a first enzyme that is methylation insensitive, for example, Taq I. The methylation insensitive enzyme may also be referred to as the “anchor enzyme”. Next, the fragmented DNA is digested with a second enzyme that is methylation sensitive, for example, HpaII. The methylation sensitive enzyme may also be referred to as the “interrogation enzyme”. The anchor enzyme and the interrogation enzyme preferably do not have the same recognition sequence. Preferably the ends generated by the two enzymes are “sticky ends” that are compatible and facilitate ligation. For example, TaqI and HpaII both generate a 5′ overhang of 5′-CG-3′. AciI is methylation sensitive and also generates a compatible single stranded overhang. After digestion with the anchor enzyme and the interrogation enzyme at least some of the fragments are circularized by ligation. Intramolecular circularization, circularization of a single fragment by ligation of the two ends of the fragment, is favored over intermolecular ligation, circularization of two or more fragments, at concentrations below the j-factor. J-factor is length dependent and is a measure of flexibility of DNA. Jacobson and Stockmayer J. Chem. Phys. 18:1600-1606 (1950) first described the J-factor. See also, Crothers et al., Methods Enzymol. 212: 3-29 (1992), Shore et al., PNAS 78:4833-4837 (1981) and Hagerman, P. J. Annu. Rev. Biophys. Biophys. Chem. 17:265-286 (1988). If the selected enzymes do not have compatible ends, a fill-in step may be necessary prior to ligation. The fill in step may be used to generate blunt ends that are compatible with ligation. In another embodiment, a linker may be used to ligate the ends together.

After ligation, an optional treatment with exonuclease may be performed to remove uncircularized fragments. Circles may then be amplified using, for example, RCA. (See, for example PCT Publication No. WO9201813 and U.S. Pat. No. 5,854,033). To identify the presence or absence of predicted ligation junctions, the amplification product is labeled and hybridized to an array that includes probes that are complementary to predicted junctions. Generation of the circles results in the juxtaposition of regions of the genome that would not normally be contiguous. These junctions can be detected by hybridization of a probe or probes that are complementary to the newly formed junction. The presence of absence of selected junctions is an indication of the presence or absence of methylated interrogation sites in the starting sample. The type of junction formed will depend on the methylation pattern. All possible junctions can be predicted based on the sequence of the genomic DNA and in silico digestion methods. The array consists of probes that hybridize to all possible junctions that can form when fragments containing one end generated by Enzyme 1 and one end generated by Enzyme 2, both ends generated by Enzyme 1, or both ends generated by Enzyme 2 are circularized. Alternatively, a subset of the junctions may be interrogated.

In another embodiment, the anchor enzyme may be omitted and the digestion performed with only the methylation-sensitive enzyme. In this case, the junction probes would consist of all combinatorial possibilities (within a chromosome) that would result by cutting with a methylation-sensitive enzyme and circularizing the resulting fragments, where the fragments are within a size range, X, where X would be, for example, 200 to 4000 base pairs. The combinatorial possibilities for probes can be reduced by making junctions for ring closures such that only junctions that could form between sites that are within a range of distances from each other would be represented on the array. The efficiency of circularization of a nucleic acid depends on the length of the nucleic acid and the concentration. Below a certain length it is difficult to circularize because of steric hindrances. Above certain lengths, for example, above 4,500 base pairs, self ligation becomes less favored. See, Shore et al. PNAS 78:4833 (1981). The value of X is to be based on experimental parameters for the maximum distance between two ends that still allows for efficient ring closure. Exonuclease treatment of large and small fragments that cannot circularize efficiently or that may not amplify efficiently because of size, would serve to reduce the complexity. In one aspect fragments that are between about 150, 200 or 500 to about 1,000, 2,000 or 4,000 base pairs circularize and are amplified. In a preferred aspect the size range is about 200 to 4,000 and in another aspect the size range is about 150 to 2,000 base pairs.

Multiple enzymes may be used at once for each restriction digest step and the probes may again be chosen based on all the possible outcomes. Alternatively, the digestion may be performed with the anchor enzyme(s) and the digestion split into several vessels for subsequent digestion with different interrogation enzymes. After digestion with the interrogation enzyme, the fragments can be circularized and amplified. If desired, the different interrogation digests can be recombined at the circularization, amplification or post-amplification stages.

The methylation insensitive enzyme selected is chosen to optimize the size of the resulting fragments for ligation, amplification and number of MS sites. Ligation conditions are preferably optimized to favor ligation of the ends of a single fragment over ligation of the ends of multiple fragments.

In one aspect the MI and MS enzymes are selected so that ligation of the ends generated by the MI (anchor) enzyme to the ends generated by the MS (interrogation) enzymes results in a new site that cannot be cleaved by the anchor enzyme. The products of the ligation reaction can then be digested with the anchor enzyme. This will linearize products formed by circularization of fragments having both ends generated by the anchor enzyme. This class of fragments represents a population that either did not have sites for interrogation by the methylation sensitive enzymes or a population in which the sites were completely methylated. Such fragments once linearized, could be eliminated by exonuclease treatment thereby reducing the complexity of the sample.

In another aspect, the enriched and amplified sample is hybridized to an array that includes probes that hybridize to the regions that flank the MS site.

In some aspects methylation at highly repetitive regions of the genome may be analyzed for methylation status by selecting anchor enzymes that generate unique junctions. In this way the probes can be complementary to the unique junction and would not need to be complementary to naturally occurring repetitive regions. This allows for analysis of low complexity regions of the genome as well.

In one aspect the methods include restriction digestion of a sample with two restriction enzymes that recognize the same recognition site but are differentially sensitive to methylation. An example of such an enzyme pair is HpaII and MspI. HpaII and MspI are isoschizomers that cleave at the recognition site CCGG. There are many enzymes that are commercially available from, for example, New England Biolabs. Information about commercially available enzymes may be found, for example, at web sites such as the New England Biolabs website or in the New England Biolabs catalogue. Cleavage by HpaII is blocked by methylation of the central C while MspI cleaves independent of methylation of the central C. In one aspect of the invention aliquots of a sample are subjected to digestion with either MspI or HpaII, in parallel, and cleavage products are analyzed and compared to determine methylation status. If the site of interest is methylated it will not be cleaved by HpaII but will be cleaved by MspI. A hybridization pattern can be obtained for each aliquot and the patterns may be compared to determine the presence or absence of methylation at selected sites.

Many of the embodiments may include one or more steps of computer implemented in silico digestion. In silico digestion typically involves analysis of the sequence of a genome or genomic region to locate the recognition sites for a selected restriction enzyme or combination of enzymes and predicting the sizes and sequences of the fragments that will result from digestion of a sample with the selected enzyme or enzyme combination. The output of the in silico digestion may be, for example, an electronic file reporting the sequence of predicted fragments. The fragments may be subsequently interrogated for other features, such as the presence of a polymorphism or the presence of another restriction enzyme recognition site. In one aspect a computer is used to identify restriction fragments that contain one or more recognition sites for a methylation sensitive restriction enzyme or a methylation dependent restriction enzyme. A computer may also be used to design probes to detect a plurality of predicted fragments and to design an array of probes. A computer may also be used to identify fragments that are amenable to amplification by the PCR conditions. In many embodiments the PCR conditions preferentially amplify fragments of a limited size range, for example, 100, 200 or 400 to 800, 1,000 or 2,000 base pairs. Fragments that are within the expected size range and contain a site for a methylation sensitive enzyme are identified and an array may be designed with probes complementary to a plurality of the fragments that are identified.

In an exemplary aspect, (FIG. 4) a sample is first digested with an enzyme that cuts outside of methylated regions, for example, XbaI. Aliquots of the digested sample are separately digested with each of a pair of isoschizomers where one is methylation insensitive and the other is methylation sensitive, for example, Msp I and Hpa II. The fragmented aliquots are then incubated with ligase to promote formation of circular fragments and then treated with exonuclease to remove linear fragments. The remaining fragments are amplified, labeled and hybridized to an array of junction probes to generate a hybridization pattern for each aliquot. The patterns are compared.

In the example shown in FIG. 4 the isoschizomers are indicated by MS (methylation sensitive) of MI (methylation insensitive). The recognition site for the enzyme pair is indicated by MS/MI. An example of such an isochizomer pair is Hpa II (MS) and Msp I (MI). In the figure, the first MS/MI site is methylated and the second is unmethylated. Both samples are hybridized to a junction array designed with probes to detect the predicted junctions. The samples may be hybridized separately to different copies of the array or differentially labeled and hybridized to the same array simultaneously.

In the first aliquot, treated with the MS enzyme, the methylated site is not digested and the junctions that are detected are a/d, e/f and g/h. Junctions a/b and c/d are not detected or may be detected at a reduced level. The second aliquot is treated with the MI enzyme and both sites are cut. The junctions detected are a/b, c/d, e/f and g/h. Junction a/d is not detected. From the analysis of the hybridization from the first aliquot the absence (or reduction) of signal from the a/b and c/d junctions and the presence of the a/d junction indicate that the first site was methylated. From the analysis of the hybridization from the second aliquot the presence of the a/b and c/d junctions and the absence of the a/d junction show the level of hybridization expected for those junctions in the absence of methylation. Preferably the level of hybridization to junction probes complementary to the a/b and c/d junctions in the sample treated with the methylation insensitive enzyme may be compared with the level of hybridization to those junction probes in the sample treated with the methylation sensitive enzyme and reduced hybridization after treatment with the methylation sensitive enzyme compared to the methylation insensitive enzyme is indicative of methylation.

In one embodiment a first aliquot of a sample is first digested with a methylation insensitive enzyme, fragments are circularized, linear fragments are digested with an exonuclease and the remaining fragments are amplified. A second aliquot of the sample is treated with a methylation sensitive isoschizomer of the first enzyme, followed by circularization, exonuclease digestion of linear fragments and amplification. A hybridization pattern is obtained for each aliquot and compared. Differences in the hybridization pattern are indicative of the methylation pattern.

In one aspect, a computer system is used to locate and map methylated fragments in the genome based on the expected products of the first fragmentation reaction and the sequence of the probe showing hybridization. In addition a computer may be used to identify CCGG sites in the identified fragment. In one aspect of the invention, the array of probes comprises probes that are complementary to regions of the genome that contain CpG islands. The probes may be designed to be complementary to a region that will be in the same restriction fragment as the CpG island, but may be complementary to a region that does not contain CpG dinucleotides.

Bisulfite Modification Based Methods

In some embodiments the methods include treatment of the sample with bisulfite. Unmethylated cytosine is converted to uracil through a three-step process during sodium bisulfite modification. The steps are sulphonation to convert cytosine to cytosine sulphonate, deamination to convert cytosine sulphonate to uracil sulphonate and alkali desulphonation to convert uracil sulphonate to uracil. Conversion on methylated cytosine is much slower and is not observed at significant levels in a 4-16 hour reaction. See Clark et al., Nucleic Acids Res., 22(15):2990-7 (1994). If the cytosine is methylated it will remain a cytosine. If the cytosine is unmethylated it will be converted to uracil. When the modified strand is copied, through, for example, extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated. When the double stranded extension product is amplified those Cs that were converted to U's and resulted in incorporation of A in the extended primer will be replaced by Ts during amplification. Those Cs that were not modified and resulted in the incorporation of G will remain as C.

Kits for DNA bisulfite modification are commercially available from, for example, Human Genetic Signatures' Methyleasy and Chemicon's CpGenome Modification Kit. See also, WO04096825A1, which describes bisulfite modification methods and Olek et al. Nuc. Acids Res. 24:5064-6 (1994), which discloses methods of performing bisulfite treatment and subsequent amplification on material embedded in agarose beads. In one aspect a catalyst such as diethylenetriamine may be used in conjunction with bisulfite treatment, see Komiyama and Oshima, Tetrahedron Letters 35:8185-8188 (1994). Diethylenetriamine has been shown to catalyze bisulfite ion-induced deamination of 2′-deoxycytidine to 2′-deoxyuridine at pH 5 efficiently. Other catalysts include ammonia, ethylene-diamine, 3,3′-diaminodipropylamine, and spermine. In some aspects deamination is performed using sodium bisulfite solutions of 3-5 M with an incubation period of 12-16 hours at about 50° C. A faster procedure has also been reported using 9-10 M bisulfite pH 5.4 for about 10 minutes at 90° C., see Hayatsu et al, Proc. Jpn. Acad. Ser. B 80:189-194 (2004).

Bisulfite treatment allows the methylation status of cytosines to be detected by a variety of methods. For example, any method that may be used to detect a SNP may be used, for examples, see Syvanen, Nature Rev. Gen. 2:930-942 (2001). Methods such as single base extension (SBE) may be used or hybridization of sequence specific probes similar to allele specific hybridization methods. In another aspect the Molecular Inversion Probe (MIP) assay may be used.

In a preferred aspect, molecular inversion probes, described in Hardenbol et al., Genome Res. 15:269-275 (2005) and in U.S. Pat. No. 6,858,412, may be used to determine methylation status after methylation dependent modification. A MIP may be designed for each cytosine to be interrogated. In a preferred aspect the MIP includes a locus specific region that hybridizes upstream and one that hybridizes downstream of an interrogation site and can be extended through the interrogation site, incorporating a base that is complementary to the interrogation position. The interrogation position may be the cytosine of interest after bisulfite modification and amplification of the region and the detection can be similar to detection of a polymorphism. Separate reactions may be performed for each NTP so extension only takes place in the reaction containing the base corresponding to the interrogation base or the different products may be differentially labeled.

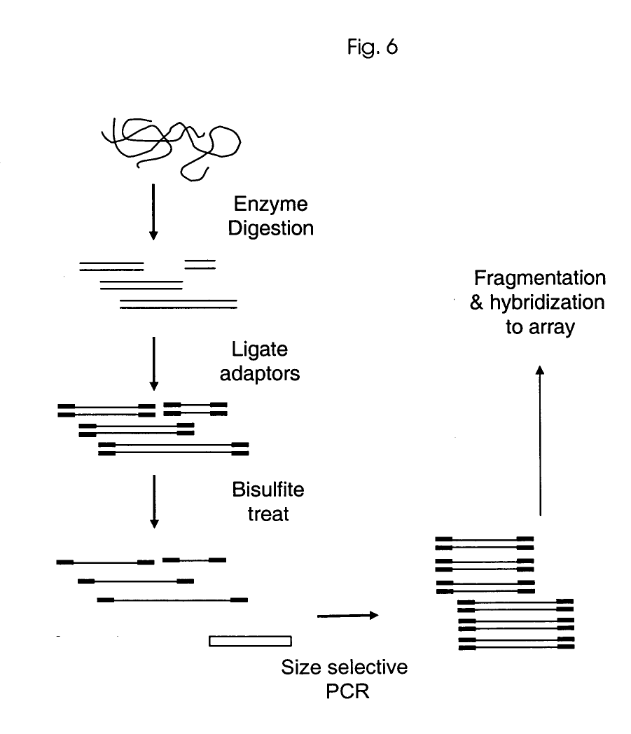

In one aspect (FIG. 6) the DNA sample is fragmented with one or more restriction enzymes and ligated to one or more adaptor sequences before treatment with bisulfite. The bisulfite treated sample may then be amplified by PCR using primers that are complementary to the adaptors. The conditions of the amplification may be selected to preferentially amplify fragments of a selected size, for example, 200 to 2000 bp, to reduce the complexity of the sample.

The bisulfite treatment may degrade the DNA so adaptors that are ligated before bisulfite treatment may be damaged or cleaved off by the treatment, making the fragments resistant to amplification. In one aspect adaptors are ligated to the DNA after bisulfite treatment. In a preferred aspect T4 RNA ligase is used for ligation of adaptors. Because the 3′ end after bisulfite treatment may be blocked from ligation adaptors may be ligated to the 5′ end (the primer may be end protected), then the 3′ end of the fragments may be treated to make it available for ligation, for example by dephosphorylation or treatment with an Endo IV, and a 5′ phosophorylated primer may be ligated to the 3′ end. Bisulfite treatment may also make the DNA single stranded because mismatches are introduced where cytosines are converted to uracils, resulting in G:U base pairs in place of G:C base pairs.

In addition to deamination of unmethylated cytosines, bisulfite treatment can result in damage to the DNA, resulting in fragmentation of the DNA. In some aspects the bisulfite treatment requires long (˜4-16 hour) incubations at a pH of about 5. During this step cytosines are sulfonated and then deamination occurs. This step also may have the unintended side effect of partial depurination of the DNA. Following deamination the sulfate groups are removed by an alkali treatment. The alkali treatment may result in strand breaks at sites where depurination has occurred. The resulting fragments can be ligated to adaptors, but it may be necessary to treat the fragments chemically or enzymatically to generate ends suitable for ligation. In some aspects alkaline hydrolysis of a depurinated site may result in a 5′ phorphorylated end that is suitable for ligation of an adaptor and a 3′ end that is not a suitable substrate for ligation because it lacks a 3′ OH. The 3′ end may be treated to remove modifications that would block ligation. In one aspect the fragments are treated with an AP endonuclease prior to ligation of adaptors. In another aspect the adaptor may be ligated to the fragments in a first reaction to ligate adaptors to the ends that are available for ligation, the reaction may then be treated to generate ends that are compatible with ligation, for example, with kinase to remove phosphates from 3′ ends or with Endo IV, and subjected to a second ligation reaction. The ends that result after depurination and chain breakage may vary depending on the mechanism of cleavage. In some aspects a 3′ phosophorylated ribose is generated, but in some aspects a mixture of ends are generated including fragments with a terminal ribose. In preferred aspects the 3′ end is chemically or enzymatically processed to create an end that is suitable for adapter ligation.

In another aspect amplification of bisulfite treated DNA may be primed with random primers, for example, random hexamers. Other methods of amplification may also be used, for example, isothermal strand displacement amplification, rolling circle amplification (Lizardi et al., Nat. Genet. 19:225-232, 1998), multiple displacement amplification (MDA) (Dean et al., Proc. Natl. Acad. Sci. 99:5261-5266, 2002) and methods such as those described in US Patent Pub Nos. 20040209298 and 20040209299. Bisulfite treatment damages the DNA and the damaged DNA may amplify poorly. Amplification methods that enable amplification of degraded samples such as those obtained from Formalin-fixed, paraffin-embedded (FFPE) samples may be used to amplify bisulfite treated DNA. Amplification methods that may be preferred for degraded samples include those methods disclosed in Wang et al., Gen. Res. 14:2357-2366, 2004 which disclosed RCA-RCA. See also, Wang et al., Nuc. Acids Res. 32:e76, 2004 which discloses methods of balanced PCR and also US Patent Pub Nos. 20040209298 and 20040209299. In a preferred aspect, the primers used for amplification are biased for bisulfite converted DNA which will have a reduced number of G/C base pairs. In the first round of amplification unmethylated cytosine will generally have been converted to uracil so the primers may be biased to have fewer or no Gs. In one aspect bisulfite treated DNA is incubated with antibodies to 5-meC or with 5-meC binding proteins and antibodies to the proteins and antibody associated complexes are isolated. The DNA from the isolated complexes may be amplified by adaptor ligation and PCR amplification as described above.

In another aspect activation-induced cytidine deaminase (AID) is used as an alternative to bisulfite treatment. AID deaminates unmethylated cytosines while methylated-CpG motifs are protected from AID-mediated deamination, see, Larijani et al., Mol Immunol. 42(5):599-604 (2005). AID treated DNA may be analyzed by the same methods bisulfite DNA is analyzed. The AID assay had the advantage that it can be performed in a short time, about 30 minutes compared to more than 12 hours for a typical bisulfite treatment, there are fewer steps than the complicated bisulfite treatment, and fewer toxic chemicals are used. In some aspects DNA may be treated with a combination of AID treatment and bisulfite treatment. This combined approach of the two methods may be used to improve the efficiency of the AID treatment but provide for shorter bisulfite treatment and reduced degradation of the DNA.

In one aspect the methylation level of a specific cytosine may be quantified. The hybridization pattern may be analyzed to measure the levels of methylation, hybridization intensity correlating with degree of methylation. For example, if a particular cytosine is methylated in 80% of the DNA in the sample the normalized intensity of the C “allele” should be about 4 fold the normalized intensity of the T “allele” after bisulfite treatment. Methods for quantifying methylation levels of specific cytosines using bisulfite treatment have been disclosed, for example, in Thomassin et al., Nuc. Acids Res. 32:e168 (2004).

In a preferred aspect the products are analyzed by hybridization to an array. In one exemplary embodiment an array is designed to detect the products of bisulfite modification using the same principles as the commercially available Affymetrix 10K Mapping Array. The 10K array has probe sets for each of more than 11,000 different human SNPs. Each probe set has a first plurality of probes that are perfectly complementary to a first allele of the SNP and a second plurality of probes that are perfectly complementary to the second allele of the SNP. If the first allele is present signal is detected by the first plurality of probes and if the second allele is present signal is detected by the second plurality of probes. Heterozygotes result in signal detection by both. The probe sets may include control probes, for example, mismatch probes, probes that shift the interrogation position relative to the central position of the probe may be included, for example, the SNP position may be at the central position or it may be shifted 1 or more positions 5′ or 3′ of the center of the probe. Analogous probe sets could be designed for suspected sites of methylation, treating the position as though it were a SNP with alleles C/G or T/A. Both strands may be analyzed. Exemplary probes and arrays are described in U.S. patent application Ser. No. 10/681,773 and U.S. Pat. Nos. 5,733,729, 6,300,063, 6,586,186, and 6,361,947. The bisulfite treatment can modify any unmethylated C in the fragments, including C's in primer binding sites and C's that are in regions surrounding an interrogation positions. In preferred embodiments the adaptors are designed to take this into account, for example, the adaptor may be designed so that there are no C's in the primer binding site, the primer may also be synthesized with modified bases that are resistant to bisulfite modification so that the sequence of the primer binding site is not changed by the treatment, for example, C's could be methylated, or the primer can be designed assuming that the C's in the adaptor will be changed to U's.

Resequencing arrays which allow detection of novel SNPs from a sequence may also be used to detect the products of the bisulfite treatment. Resequencing arrays and resequencing methods are described, for example, in Cutler et al. Genome Res. 2001 November; 11(11):1913-25 and in US patent publication No. 20030124539, both of which are incorporated herein by reference in their entirety. In general resequencing arrays detect all possible single nucleotide variations in a reference sequence. Probes are included that are perfectly complementary to the reference sequence and interrogate a plurality of positions in the sequence individually for variation in the reference sequence. Probes that are perfectly complementary to the variant sequence are included for each possible variation. An array may be tiled to detect all possible single nucleotide variations in one or more reference sequences. To detect the products of bisulfite treatment, instead of designing probes to all possible single nucleotide variants, the probes may be designed to detect possible variations at cytosines, depending on methylation. The reference sequence or sequences interrogated by the array may be, for example, one or more entire chromosomes, one or more entire genomes, one or more mitochondrial genomes, or selected regions of interest from within one or more genomes. In one embodiment a resequencing array is tiled with regions that are known or suspected to be methylated. In some embodiments CpG sites may be close together so that the probes of the array may be complementary to overlapping CpG sites. For example if the probe is a 25 mer and the interrogation position at position 13 is complementary to a first cytosine position there may be a second CpG that is within the 12 base pairs upstream or the 12 base pairs downstream of the first cytosine. The second cytosine may or may not be methylated. Probes can be designed to detect both possibilities, i.e. both methylated (both C), both unmethylated (both T), one methylated (C) and the other unmethylated (T). Probes that are perfectly complementary to each possible outcome may be designed.

In another aspect of the invention amplified methylated target is enriched relative to unmethylated target. In one exemplary embodiment a nucleic acid sample suspected of containing 5-meC is fragmented using a restriction enzyme and adaptors are ligated to the fragments. Antibodies to 5-meC are used to isolate adaptor-ligated fragments that contain 5-meC. Alternatively the nucleic acid may be incubated with proteins that specifically bind 5-meC and then antibodies to those proteins may be used to isolate methylated fragments. Antibodies to 5-meC are available, for example, ab1884 available from Abcam (Cambridge, UK). The isolated fragments are amplified by PCR using a primer complementary to the adaptor and the amplified fragments may be hybridized to an array of probes. In a preferred aspect the probes of the array are complementary to one or more regions of the genome. Regions of the array that show hybridization above background are indicative of areas of the genome that are methylated. In a preferred embodiment the array comprises probes to CpG rich regions of the genome, intragenic regions, or regions known or predicted to be regulatory regions. In another embodiment the immunoprecipitated fragments are treated with bisulfite so that precise locations of methylated cytosines may be identified. The sample may be analyzed by hybridization to an array of sequence specific probes as described above.

In one aspect of the invention methyl binding proteins, such as MeCP2 and SAP18/30 (Sin3 associated Polypeptides 18/30), are mixed with the genomic DNA sample and used to enrich for methylated sequences. Antibodies to methyl CpG binding domain proteins (MBDs), for example, MBD2 and MBD3 may be used to isolate DNA containing methylation. Antibodies against 5-meC-binding proteins are available, for example, antibodies to MeCP2 (IMG-297) are available from Imgenex Corp. (San Diego, Calif.). In another aspect antibodies that recognize 5-meC may be used to enrich for methylated sequences. The DNA is preferably denatured prior to antibody binding.

In another aspect of the invention methylation is used as a means of separating a genome into subsets in a relatively reproducible manner in order to reduce the complexity of the sample prior to further analysis. Some regions of the genome are stably methylated while other regions are stably unmethylated. Mechanisms that differentiate between methylated and unmethylated DNA can be used to obtain fractions of a sample that are enriched for either methylated or unmethylated DNA. In this way the complexity of a sample can be reduced. Separation may be prior to if amplification is by a method that maintains methylation information. It is often desirable to reduce the complexity of a sample that contains a complex mixture of nucleic acids prior to hybridization to improve sensitivity of detection and minimize background.

In one aspect of the invention methods for analyzing nucleic acid samples following separation of methylated and unmethylated fractions are disclosed. The fraction that is analyzed may be the methylated or unmethylated fraction or a comparison of methylated and unmethylated fractions may be made. In many embodiments the unmethylated fraction is enriched, for example through separation of methylated DNA from unmethylated or by preferential amplification of unmethylated DNA. Isolation of a fraction that is enriched for a subset of the starting nucleic acids may be used as a method of reducing the complexity of a sample or as a method of measuring differences between the methylated fraction and the unmethylated fraction. In one embodiment the methods are particularly useful for analyzing a sample to identify regions of the genome that are present in the unmethylated fraction and regions of the genome that are present in the methylated fraction. In many embodiments the methods for separation of methylated and unmethylated nucleic acids are combined with methods of analysis of nucleic acids with arrays of probes.

In one aspect CpG islands are enriched by digesting the DNA sample with an enzyme, such as MseI followed by size selection. MseI has a 4 base pair recognition site that includes only A's and T's. MseI cuts genomic DNA into small fragments but cuts infrequently in CpG islands. The larger fragments, enriched for CpG islands, may be separated from the smaller fragments by any available size separation method, for example, size exclusion chromatography or electrophoretic methods. Other 4 cutter enzymes that don't have CpGs in their recognition site may also be used. A combination of enzymes may also be used.

Reduced complexity samples that are a representation of a more complex sample, such as a genome can be used for a variety of analysis methods, including those that involve hybridization. Reduced complexity samples may be used, for example, for sequencing applications, genotyping, quantitative assessment of copy number, LOH analysis, and CGH analysis. In many embodiments the analysis is by hybridization of the reduced complexity sample to an array of probes. Arrays for expression analysis, resequencing, and genotyping, for example, are available from Affymetrix, Inc., Santa Clara, Calif.

Methods for separation of methylated from unmethylated nucleic acids have been described, see, for example, US patent publication nos. 20010046669, 20030157546, and 20030180775 which are each incorporated herein by reference in their entireties.

Repetitive sequences in plant and mammalian genomes are often present in high copy number, have high levels of cytosine and low transcriptional activity (See, e.g., Martienssen, R. A. (1998) Trends Genet. 14:263; Kass, S. U., et al. (1997) Trends Genet. 13:335; SanMiguel, P., et al., (1996) Science 274:765; Timmermans, M. C., et al. (1996) Genetics 143:1771; Martienssen, R. A. and E. J. Richards, (1995) Curr. Opin. Genet. Dev. 5:234-242; Bennetzen, J. L., et al. (1994) Genome 37:565; White, L. F., et al. (1994) Proc. Natl. Acad. Sci. U.S.A. 91:11792; Moore, G., et al. Genomics 15:472). High copy DNA sequences are frequently methylated and often are not present in areas of expressed genes. Methods that eliminate or reduce the representation of such high copy methylated DNA from a library or from a nucleic acid sample may be used to enrich for target sequences of interest and result in a sample that has a complexity that is reduced, facilitating further analysis. Often the unmethylated regions are the regions that contain the genes and are of the highest interest for analysis.

Nucleic acid samples may be enriched for sequences that are unmethylated by propagation of nucleic acid libraries, for example genomic libraries which may be partial libraries, in methylation restrictive hosts, such as E. coli strains JM101, JM107 and JM109. This method, methylation filtration, was recently used to sequence the genome of maize, see Palmer et al. Science 302:2115-2117 (2003). The method prevents the propagation of clones carrying methylated inserts, resulting in the enrichment of genes five to sevenfold when compared to control libraries.

In another embodiment nucleic acid samples are digested with enzymes that are methylation sensitive, for example enzymes that cleave only unmethylated DNA or cleave only methylated DNA or methylation insensitive enzymes that cleave methylated or unmethylated DNA. Differentially digested samples may be amplified and the amplified fragments may be labeled and then detected using microarrays. A sample may be digested in parallel with a methylation sensitive enzyme and a methylation insensitive enzyme and analyzed to determine which sequences are present following each treatment. Sequences that are present in the first sample but not the second sample indicate that the sequence was methylated.

In one exemplary embodiment a nucleic acid sample is obtained from a source, such as from an individual, the nucleic acid may be fragmented, for example by digestion with one or more restriction enzymes, and an adaptor sequence may be attached to the fragments to generate adaptor-ligated fragments. The adaptor-ligated fragments may be digested with an enzyme that cleaves methylated DNA but not unmethylated DNA, for example, McrBC. The sample may then be amplified with a primer that hybridizes to the adaptor sequence. The methylated fragments that have been cut with the methyl specific enzyme are not amplified because they have the adaptor only on one end, resulting in selective amplification of unmethylated DNA.

The amplified products may be detected by, for example, hybridization to a microarray. The McrBC digested sample may be compared with a parallel sample that was not digested with McrBC to identify regions that were methylated. If the products are hybridized to an array of probes in parallel, probes to the regions that were methylated in the sample should show hybridization in the sample that was not digested with McrBC but not in the sample that was digested with McrBC. Because the presence of methylation in the fragment is detected by detecting the presence or absence of the restriction fragment there is considerable flexibility in the design of the probes that would be suitable. For example, the fragments to be detected will typically be between 200 and 1,000 base pairs and probes may be targeted to any region of the fragment. Probes need not be complementary to the site of methylation but can be complementary to a site upstream or downstream. Probes may be targeted to one region of the fragment or a plurality of regions in the fragment, they may be targeted to a specific feature of the fragment, for example, a SNP in the fragment or to one or more CpG's in the fragment. In one embodiment an array of probes comprising probes spaced evenly throughout the genome may be used.

In an exemplary embodiment the amplified products are labeled and hybridized to a genotyping array, for example, the Mapping 10K or 100K Array (Affymetrix, Santa Clara). The GeneChip Mapping Assay (WGSA) may be used to reduce the complexity of a sample. The basic steps of the assay are as follows: total genomic DNA (250 ng) is digested with a restriction enzyme (e.g. XbaI) and ligated to adaptors that recognize the cohesive four basepair overhangs. All fragments resulting from restriction enzyme digestion, regardless of size, are substrates for adaptor ligation. A generic primer that recognizes the adaptor sequence is used to amplify adaptor ligated DNA fragments. PCR conditions that are optimized to preferentially amplify fragments of a selected size range (e.g. 250 to 2000 bp) are used for amplification. Conditions may be optimized to select for different size ranges, for example 200 to 1,000 base pairs. The amplified DNA is then fragmented, labeled and hybridized to the Mapping 10K Array. The probes of the array are selected to be complementary to regions of the genome that are predicted by in silico digestion to be present on fragments of the selected size range (e.g. 250 to 1000 bp when the genome is digested with XbaI). In this way the amplification enriches for a subset of fragments, the same subset of fragments is reproducibly enriched and the array is designed to interrogate at least some of those fragments. The Mapping 10K and 100K Array interrogates the genotype of known SNPs present on the predicted fragments, but in other embodiments an array may be designed to interrogate for the presence or absence of a fragment. For additional information about the Mapping 10K array and assay see the GeneChip Human Mapping 10K Array and Assay Kit Data Sheet, part no. 701366 Rev. 4, Affymetrix, Inc. and the Mapping 10K Manual.

In one embodiment arrays that comprise probes that are complementary to genes in an organism, may be used to analyze methylated or unmethylated fractions. For example, expression arrays available from Affymetrix, such as the Human Genome U133 Plus 2.0 array, may be used. Expression arrays are available for a number of organisms including Mouse and Rat and can be custom designed for an organism of choice. Arrays comprising probes to predicted or known exons, or splice junctions (intron-exon or exon-exon) may also be used.

In one embodiment high density arrays that tile an entire genome, one or more entire chromosomes or a representation of an entire genome or one or more entire chromosomes may be used to analyze a sample prepared by separation of methylated and unmethylated DNA. For example, an array that contains probes spaced on average every 35 base pairs along one or more chromosomes or an entire genome may be used. See, for example, Kapranov et al. Science 296:916-919 (2002). See also U.S. patent application Ser. Nos. 10/741,193, 10/736,054, 10/714,253, and 10/712,322. In one embodiment a sample that has been enriched for unmethylated sequences may be analyzed by transcription factor binding affinity. Sequences that bind to transcription factors may be purified by affinity to transcription factors and then identified by array analysis. Complexity may similarly be reduced by enrichment for methylated sequences, by digestion with enzymes that cleave only unmethylated DNA.

A number of methyl-dependent restriction enzymes are known to those of skill in the art and are available commercially from, for example, New England Biolabs. Examples of methyl-dependent restriction enzymes include, McrBC, McrA, MrrA, and DpnI. McrBC is an endonuclease which cleaves DNA containing methylcytosine, (e.g. 5-methylcytosine or 5-hydroxymethylcytosine or N4-methylcytosine, reviewed in Raleigh, E. A. (1992) Mol. Microbiol. 6, 1079-1086) on one or both strands. McrBC will not act upon unmethylated DNA (Sutherland, E. et al. (1992) J. Mol. Biol. 225, 327-334). The recognition site for McrBC is 5′ . . . Pu^{m}C(N₄₀₋₃₀₀₀) Pu^{m}C . . . 3′. Sites on the DNA recognized by McrBC consist of two half-sites of the form (G/A)^{m}C. These half-sites can be separated by up to 3 kb, but the optimal separation is 55-103 base pairs (Stewart, F. J. and Raleigh E. A. (1998) Biol. Chem. 379, 611-616 and Panne, D. et al. (1999) J. Mol. Biol. 290, 49-60.). McrBC requires GTP for cleavage, but in the presence of a non-hydrolyzable analog of GTP, the enzyme will bind to methylated DNA specifically, without cleavage (Stewart, F. J. et al. (2000) J. Mol. Biol. 298, 611-622). Recombinant McrBC is available from, for example, New England Biolabs. McrBC may be used to determine the methylation state of CpG dinucleotides. McrBC will act upon a pair of Pu^{m}CG sequence elements, but will not recognize Hpa II/Msp I sites (CCGG) in which the internal cytosine is methylated. The very short half-site consensus sequence (Pu^{m}C) allows a large proportion of the methylcytosines present to be detected.

In one embodiment reaction conditions for digestion with McrBC are 50 mM NaCl, 10 mM Tris-HCl, 10 mM MgCl₂, 1 mM dithiothreitol (pH 7.9 at 25° C.) with 100 μg/ml BSA and 1 mM GTP. Incubate at 37° C. Conditions may be varied. NEB defines one unit as the amount of enzyme required to cleave 1 μg of a plasmid containing a single McrBC site in 1 hour at 37° C. in a total reaction volume of 50 μl. A 5 to 10-fold excess of enzyme may be used for cleavage of genomic DNA. The enzyme may be heat inactivated by heating to 65° C. for 20 minutes. McrBC makes one cut between each pair of half-sites, cutting close to one half-site or the other, but cleavage positions are distributed over several base pairs approximately 30 base pairs from the methylated base. See also, Bird, A. P. (1986) Nature 321, 209-213 and Gowher, H. et al. (2000) EMBO J. 19, 6918-6923.

Studies on or utilizing McrBC have been reported in the literature, for example, Gast et al. Biol. Chem. 378(9):975-82, (1997), Pieper et al., Rabinowicz, Methods Mol. Biol. 236:21-36 (2003), Badal et al. J. Virol. 77(11):6227-34 (2003) and Chotai and Payne, J Med Genet. 35(6):472-5 (1998). See also, Lyko, F. et al. Nat. Genet., 23, 363-366 (2000) which used McrBC as a tool for enrichment of undermethylated DNA in drosophila.

In one aspect, genomic DNA is divided into a methylated fraction and an unmethylated fraction by any method known in the art. Each fraction may be separately hybridized to an array or each fraction may be labeled with a differentially detectable label, for example different colors of fluorescent dye (for example, unmethylated DNA may be labeled with green and methylated DNA may be labeled with red) and then both may be hybridized to the same array of probes. See for example, U.S. Pat. No. 6,576,424, which is incorporated herein by reference. If a region of the genome was not methylated then the feature or features corresponding to that region of the genome will be detected as green. If the region is methylated then the feature should be detected as red. If both red and green are detected the region may have been partially methylated in the sample, a ratio of red to green may be used to determine the extent of methylation.

In one aspect the disclosed methods are used to obtain a methylation signature or profile of a tumor or tissue. Methylation is of particular interest in the diagnosis, treatment and outcome prediction for cancer, see Jones and Baylin, Nat. Rev. Genet. 3:415-428 (2002) and Bird, Genes Dev. 16:6-21 (2002). Patterns of methylation may be associated with specific tumors. Samples from a specific type of tumor may be isolated and analyzed using the methods disclosed to obtain a methylation pattern characteristic of a tumor type or the stage of a tumor. In one embodiment a sample from an individual or from a tumor may be compared to the methylation pattern of a tumor of known type or stage to determine if the unknown sample is similar to one or more of the known tumor types in methylation pattern. Patterns obtained according to the methods may be used to diagnose disease, stage disease, monitor treatment, predict treatment outcome, and monitor disease progression. In many embodiments analysis is performed by a direct comparison of a hybridization pattern without correlation of the pattern to the presence or absence of any specific sequence. Differences or similarities between a pattern obtained from an unknown sample that is being analyzed and patterns obtained from known samples can be used to determine if the unknown is likely to match the known sample in methylation pattern.

In one embodiment blood samples are analyzed to detect changes in the methylation pattern of tumor cells that are sloughed-off into the blood stream. Patterns of aberrant methylation or demethylation that are characteristic of a tumor type may be identified by analysis of a blood sample. Aberrant methylation patterns may be correlated with cancer, imprinting defects and aging. In one exemplary embodiment the sample is fragmented with a first restriction enzyme and the fragments are ligated to adaptors. The adaptor-ligated fragments are then digested with an enzyme that is methylation dependent or methylation sensitive. The adaptor-ligated fragments that are not digested are amplified by PCR using a primer to the adaptor. The products of the PCR amplification are hybridized to an array of probes to generate a hybridization pattern. The hybridization pattern may be compared to a hybridization pattern from another sample that has been similarly treated. Differences between hybridization patterns are indicative of differences in the methylation patterns between the two samples. A data base of hybridization patterns that are characteristic of disease states, normal states, or tissue types may be generated and used to compare hybridization patterns of unknown samples to identify similar patterns. See, for example, U.S. Pat. No. 6,228,575 which discloses methods of sample characterization based on comparison of hybridization pattern. A variety of arrays may be used for this purpose and it is not necessary that the array be specifically designed to detect specific genomic sequences from the organism being analyzed.

In one embodiment enrichment of unmethylated DNA is combined with comparative genomic hybridization (CGH) to analyze tumor cells to identify differences between tumor DNA and normal DNA. See, for example, Kallioniemi et al. Methods 9(1):113-121 (1996). Equal amounts of differentially labeled tumor DNA and normal reference DNA, (one may be labeled with biotin and the other with digoxigenin, for example), may be hybridized to an array of probes, the signal intensities quantified, and signals that are over or underrepresented in tumor versus normal can be quantified; In one embodiment methods of analysis of methylation status may be combined with methods of estimating copy number of one or more regions of a genome. Many cancers are associated with increases in the copy number of one or more regions of the genome. Increased copy number can be detected by hybridization to arrays. The increase of copy number is detected as an increase in the intensity of hybridization. Methods for analysis of copy number using oligonucleotide arrays are disclosed, for example, in U.S. Patent Pub. No. 20040157243 which discloses specific computer methods to perform copy number analysis using, for example, the Affymetrix 10K Mapping Array and Assay.

In another aspect methods of complexity reduction that employ separation of fractions based on the presence or absence of methylation are used to enrich for sequences of interest in a sample that is a mixture of host and a pathogen genomic DNA. Some organisms lack 5-meC modifications in their genomes or have reduced levels of 5-meC. For example, pathogens such as mycoplasma have an absence of 5-meC or very low levels. For additional examples see, for example, Razin and Razin, NAR 8:1383-1390 (1980). The unmethylated pathogen DNA may be enriched by digesting the sample with a methyl dependent enzyme such as McrBC. Unmethylated pathogen DNA may also be enriched by depletion of methylated DNA using antibodies to 5-meC or 5-meC binding proteins in combination with antibodies to the binding proteins. In one aspect the sample is first fragmented with a restriction enzyme that does not have CpG in its recognition site and adaptors are ligated to the fragments. The adaptor-ligated fragments are digested with a methylation dependent enzyme so fragments that are methylated and contain the enzyme recognition site are fragmented. The adaptor-ligated fragments that were not fragmented by the methylation dependent enzyme are amplified by PCR using a primer to the adaptor. This results in an amplification product that is enriched for unmethylated DNA relative to methylated DNA.

In one embodiment methods of reducing the complexity of a genomic sample using methods that result in preferential amplification of unmethylated nucleic acids may be used to enrich for pathogen DNA in a complex mixture. For example, if a nucleic acid sample is isolated from a patient who is thought to be infected with a pathogen, the nucleic acid sample may contain a mixture of the patient's DNA and the pathogen's DNA. Many prokaryotic pathogens have lower levels of methylation than the organisms that they infect so treating the mixed sample with enzymes that preferentially degrade methylated DNA prior to amplification may be used to enrich the pathogen DNA relative to the host DNA. The amplified sample may then be analyzed to detect the pathogen DNA by, for example, hybridization to an array of nucleic acid probes. Potential interfering effects due to the presence of the host DNA are reduced allowing for improved detection of the pathogen DNA.

In one embodiment a nucleic acid sample that is suspected of containing pathogen DNA is fragmented to produce fragments and adaptors are attached to the ends of the fragments. The adaptor modified fragments are then treated with an enzyme that cleaves methylated DNA but not unmethylated DNA, for example McrBC. Fragments that contain a recognition site for McrBC will be cleaved into smaller fragments that have the adaptor sequence on only one end. The sample is then amplified by PCR using a primer or primers that are complementary to sequence in the adaptors. Fragments that were cleaved by McrBC will not be amplified because they have an adaptor and therefore a priming site at only one end. Because the pathogen sequence is not methylated it will not be cleaved by McrBC and will be amplified.

Arrays that may be used for detection of methylation include, for example, tiled arrays, arrays that have probes that are perfectly complementary to a plurality of possible combinations of CpG and 5-meCpG after bisulfite treatment for the region of interest. Methylation may be analyzed on both strands or on one strand. If probes are designed to one strand they may be designed to interrogate either strand. Choice of strand to be interrogated in some aspects is the strand containing the cytosine, while in other aspects, that strand has been amplified after modification so that the resulting amplified double stranded product has an A:T basepair in place of the C:G base pair and either strand can be interrogated. All unmethylated cytosines may be converted to uracils and probes and primers may be designed to take this into account, for example, probe locations that are complementary to positions that are cytosines in the genomic sequence should have A's in the position that is complementary to the cytosine position.

Exemplary arrays that may be used in combination with the disclosed methods include the arrays disclosed in U.S. patent application Ser. Nos. 09/916,135 and 10/891,260 and U.S. Patent Pub. No. 20040067493, each of which is incorporated herein by reference.

A database of junction probes that are methylation informative may be obtained by modeling restriction digestion with different combinations of methylation sensitive and methylation insensitive enzymes. A computer may be used to model fragmentation of a genome or a genomic region using a first methylation insensitive restriction enzyme (RE1). The digestion of the RE1 fragments with a methylation sensitive enzyme (RE2) is then modeled, assuming different methylation possibilities. Each RE2 site may be either methylated or unmethylated and this will determine whether the site will be cut by RE2. The modeling may take into account all possible variations on RE2 methylation, for example, if a RE1 fragment has two RE2 sites (RE2-1 and RE2-2) the possible combinations for methylation are: both sites methylated or both unmethylated, the RE2-1 methylated and the RE2-2 not methylated or the RE2-1 not methylated and the RE2-2 methylated. Each possible methylation combination should result in the formation of different predictable junction sequences after ligation. A computer can be used to compare the results of hybridization to an array of junction probes to the expected database of modeled junctions to determine which RE2 sites were methylated and which were unmethylated.

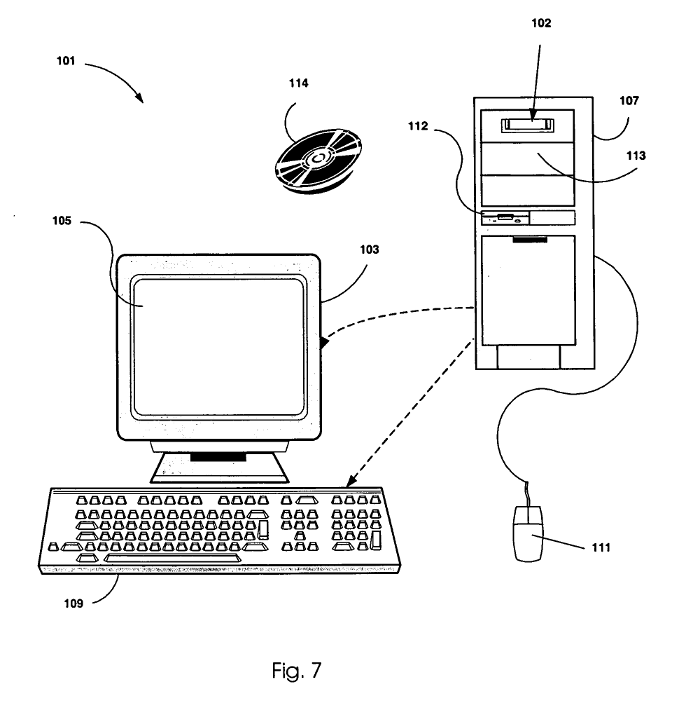

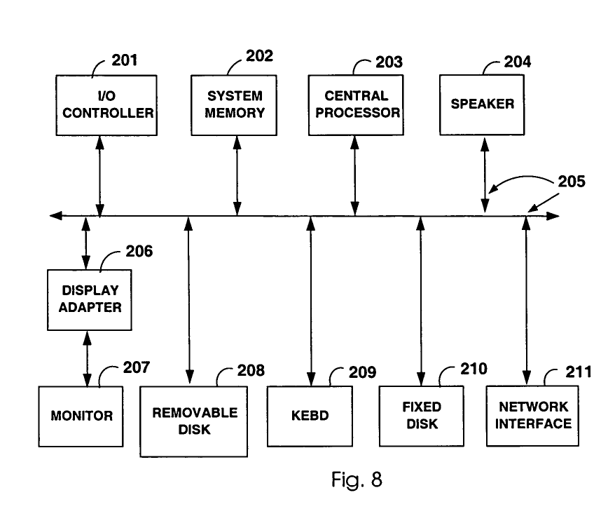

FIG. 7 shows a computer system 101 that includes a display 103, screen 105, cabinet 107, keyboard 109, and mouse 111. Mouse 111 may have one or more buttons for interacting with a graphic user interface. Cabinet 107 houses a floppy drive 112, CD-ROM or DVD-ROM drive 102, system memory and a hard drive (113) (see also FIG. 8) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 8 shows a system block diagram of computer system 101 used to execute the software of an embodiment of the invention. As in FIG. 7, computer system 101 includes monitor 201, and keyboard 209. Computer system 101 further includes subsystems such as a central processor 203 (such as a PENTIUM® processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument. In a preferred aspect computer readable code

In a preferred aspect, the probes of the junction array may be 25 nucleotides in length and may be tiled from −4 to +4 or from −3 to +3. If the junction is: ttctttgtgatgtgtactttcatctcacag (SEQ ID NO: 1) with the restriction site in bold and underlined an example of the probes for a −3 to +3 tiling is as follows (shown 3′ to 5′ left to right):

| | |
|---|---|
| 3′ gacactctactttcatgtgtagtgt 5′ | (SEQ ID NO. 2) |
| 3′ acactctactttcatgtgtagtgtt 5′ | (SEQ ID NO. 3) |
| 3′ cactctactttcatgtgtagtgttt 5′ | (SEQ ID NO. 4) |
| 3′ actctactttcatgtgtagtgtttc 5′ | (SEQ ID NO. 5) |
| 3′ ctctactttcatgtgtagtgtttct 5′ | (SEQ ID NO. 6) |
| 3′ tctactttcatgtgtagtgtttctt 5′ | (SEQ ID NO. 7) |

Probes for a single strand are shown but probes for both strands may be included on the array. Junction probe sets may be included for all junctions that could form by unimolecular ligation assuming complete cleavage by the methylation insensitive enzyme. The array may be designed to interrogate only a subset of junctions. Some junctions may be selectively left off the array, for example, probe sets for junctions that result from the ligation of the ends of very small or very large circles may be selectively eliminated from the array design. Probe sets for junctions of circles that do not have any CpGs, circles that lack sites for methylation sensitive enzyme or circles that lack an McrBC site may also be eliminated from the array design. Alternatively a subset of junctions that are of particular interest may be selected for interrogation.

The junction array may be designed and synthesized using standard methods. After hybridization of the sample the hybridization pattern may be analyzed. The hybridization pattern from a sample enriched for methylated DNA (111 in FIG. 1) or from a sample enriched for unmethylated DNA (113 in FIG. 1) may be compared to the pattern resulting from hybridization of the amplification of the total sample after circularization (110 in FIG. 1). A decrease in the ratio of signal of enriched methylated to total for a selected junction probe set, for example, indicates unmethylated CpGs. Comparisons can also be made between samples, for example, tumor versus normal control.

For sites that can be cut by methylation sensitive and insensitive isoschizomers, the total circle population can be cut with either enzyme (in separate reactions). When the DNA is digested with methylation sensitive enzymes, sites which are partially methylated would have signals that are intermediate between uncut and fully cut (digested with the methylation insensitive isoschizomer) samples. Fully methylated sites would have signal similar to uncut.

As a normalization control, junctions may be included for circles that have no CpGs or no recognition sites for the methylation sensitive enzymes; ideally, fragments from all size ranges would be included. This population is not expected to change as a function of treatment with methylation sensitive enzymes and/or McrBC. Thus they may be used for signal normalization. McrBC has also been reported to cleave circular fragments containing only a half recognition site for McrBC.

Amplified DNA that is enriched in either methylated or unmethylated sequences can be hybridized to tiling arrays. The total population of circles amplified by REPLIg or total genomic DNA may be used as a control. Analysis may be done, for example, using methods similar to those described by Cawley et al., Cell, 116:499-509, (2004). This can be carried out using Affymetrix GTAS software. This approach may also be used to analyze data generated from the method shown in FIG. 2. In another aspect analysis may be performed by median normalizing data sets. For each predicted fragment, the probe set is defined as all probes that interrogate a particular fragment. The probe set intensity is determined. Probe set intensity ratios of treated to untreated can be used to determine whether a particular fragment is enriched or depleted.

In one aspect an array is designed to interrogate methylation status of more than 50,000, more than 100,000, more than 500,000, more than 1,000,000, more than 2,500,000 or more than 5,000,000 of these CpG's. In some embodiments the array may also contain probes to interrogate CNG positions which can also be methylated at the cytosine. Interrogation may be, for example, analogous to detecting a polymorphism at the cytosine position, reflecting the change of the cytosine to a uracil by either chemical, for example bisulfite, or enzymatic, for example AID, mechanisms. Particular CpG's may be selected for interrogation based on the positioning of neighboring CpG dinucleotides. When there are more than one CpG in the region that the probe is complementary to, for example, within the 25 bases of the probe, the perfect complementarity of the probe to interrogate the central CpG may be impacted by the methylation status of the second, third or fourth CpG within the probe region. In some aspects the probe set for interrogation of the first CpG (the interrogation CpG) may be designed to take in all possible combinations of sequence variation resulting from variation in the methylation status of the secondary (non-interrogation) CpGs. This would require additional probes for each possible sequence variation. In another aspect CpGs that do not have another CpG within 12, 15, 20 or 30 bases upstream or downstream are selected for interrogation.

In another aspect, the disclosed methods may be used to detect epigenetic changes in cells that are being grown in cell culture. Cell lines that have been grown in cell culture for many generations may develop epigenetic changes that may alter the expression or growth of the cells, potentially making the cells more prone to formation of tumors, for example. The disclosed methods may be used to analyze cells in culture, for example, cell lines derived from embryonic stem cells to identify epigenetic changes that may impact the usefulness of the cultured cells. The methods may be used for quality control for cell culture.

### EXAMPLE

Methylation Based Enrichment by Fragmentation and Circularization

Step 1: ApoI Digest

Mix 20 μl 10× NEB Buffer #3, 2 μl 100×BSA, 7.14 μl genomic DNA (1.4 μg/μl), 160.86 μl H₂O and 10 μl ApoI. Mix the reaction well by flicking the tube. Spin down briefly. Reaction can be split into two tubes. Incubate at 50° C. for 6 hours in a PCR machine with heated lid. Heat inactivate at 80° C. for 20 minutes. Store at −20° C.

Step 2: Circularization.

Take 35 μl of the ApoI digest from above and incubate at 45° C. for 10 min. Snap chill on ice. Thaw ligase buffer. Keep on ice. Make 1× ligation buffer by putting 1406.1 μl of water in a new tube and chilling to 16° C. Add 160 μl of 10× ligation buffer to the water and return to 16° C. Briefly spin the digested DNA and add 32 μl to the 1× ligation buffer. Mix well, spin briefly and return to 16° C. Add 6.4 μl of ligase (2000 u/ul). Mix well by inverting the tube several times. Incubate at 16° C. for 15 minutes. Heat inactivate at 65° C. for 15 minutes. Clean up the DNA on a Qiagen PCR column. Elute in 100 μl.

After circularization an aliquot of the sample may be enriched for methylated circles or for unmethylated circles.

For enrichment of methylated circles the following protocol may be used.

Step 1: Digest with Hin6I and HpaII:

Mix 30 μl 10× Tango Buffer, 0.5-1 μg of circularized DNA (or use entirety of 100 μl from ligation step), 10 μl HpaII (10 U/μl from Fermentas), 10 μl Hin6I (10 U/μl from Fermentas) and H₂O to 300 μl. Incubate at 37° C. for 8 hours. Heat inactivate for 20 minutes at 65 C.

Step 2: Digest with either SsiI or a AciI (SsiI and AciI are Isoschizomers both Recognizing 5′-C^{v}CGC and both blocked by methylation of CpG).

For AciI: Desalt the digest in a Biospin 6 column or a microcon 30. To 300 μl of desalted DNA, add 35 μl of NEB #3 buffer, 4 μl H₂O and 11 μl of AciI (10 U/ul). For SsiI: add 70 μl of 10× Tango buffer to the Hin6I/HpaII digest, 119 μl H₂O and 11 μl SsiI. This gives a total of 500 μl reaction volume with a 2× concentration of Tango buffer.

Incubate at 37 C for 8 hours or overnight. Heat inactivate at 65° C. for 20 minutes if desired. Clean up digest on a Qiagen column. Elute in 100 μl EB buffer and store at −20 C.

For enrichment of unmethylated circles the following protocol may be used:

Mix 0.5-1 μg of DNA (or use entirety of 100 μl from ligation step), 20 μl 10×NEB Buffer #2, 2 μl 100×BSA, 2 μl 100×GTP, 10-20 μl McrBC and H₂O to 200 μl. Incubate at 37 C for 8 hours. Heat inactivate at 65 C for 20 minutes if desired. Clean up digest on a Qiagen column. Elute in 100 μl EB buffer. Store at −20 C.

The enriched circles, enriched for either methylated or unmethylated circles can then be digested with exonuclease to remove linear fragments. To digest with exonuclease, mix 112.5 μl H₂O, 25 μl 10× exonuclease buffer, 100 μl circularized DNA (adjust volume if necessary), 10 μl lambda exonuclease (5 U/μl) and 2.5 μl exonuclease I (20 U/μl). Incubate at 37 C for 1 hr. Heat inactivate 20 minutes at 80 C. Clean up on a Qiagen column and quantify yield. If desired, the protocol can be streamlined by addition of exonuclease directly to the heat inactivated restriction digest.

The circles may be amplified by random priming using strand displacing DNA polymerase. Perform the amplification according to the instructions provided with the REPLI-g kit (Qiagen, Valencia Calif.). For details of the protocol see the REPLI-g Handbook from Qiagen, January 2005, which is incorporated herein by reference. Use at least 10 ng DNA per reaction. Do at least eight 50 μl reactions per sample. As a control, include a circularized control that was not digested with either methylation-sensitive enzymes or McrBC but was treated with exonuclease.

Fragment the sample, end label with TdT and DLR and hybridize to an array according to standard protocols available from Affymetrix, Inc.

### CONCLUSION

Methods of analyzing DNA to determine the methylation status of a plurality of cytosines in the genome are disclosed. In preferred aspects the methods include steps of fragmentation, circularization and enrichment of circles with either methylated or unmethylated sites, and detection of sequences in the enriched fraction by hybridization to an array of probes.

The above description is illustrative and not restrictive. Many variations of the invention will become apparent to those of skill in the art upon review of this disclosure. The scope of the invention should, therefore, be determined not with reference to the above description, but instead be determined with reference to the appended claims along with their full scope of equivalents.

We claim:

1. A method for determining the methylation status of a plurality of cytosines in a sample comprising genomic DNA, said method comprising:
(a) fragmenting the sample with a first restriction enzyme that is insensitive to the methylation status of cytosines and a second restriction enzyme that is sensitive to the methylation status of cytosines to generate a first collection of fragments;
(b) adding a ligase to the first collection of fragments so that the ends of fragments are ligated together to form circular fragments, thereby generating a second collection of fragments;
(c) digesting linear fragments in the second collection of fragments;
(d) amplifying circular fragments in the second collection of fragments to generate a third collection of fragments;
(e) hybridizing the third collection of fragments to an array to obtain a hybridization pattern; and
(f) determining the methylation status of selected cytosines by analyzing the hybridization pattern.

2. The method of claim 1 wherein the array comprises probes that are complementary to junctions formed by circularization of fragments in step (b).

3. The method of claim 1 wherein the first enzyme is Taq I and the second enzyme is selected from the group consisting of AciI, SsiI, HapII, Hin6I, HinPI and HpaII.

4. The method of claim 1 wherein cleavage of DNA with the first restriction enzyme generates an overhang that is complementary to the overhang resulting from cleavage with the second restriction enzyme.

5. The method of claim 1 wherein the amplification in step (c) comprises rolling circle amplification using a strand displacing polymerase.

6. The method of claim 5 wherein the strand displacing enzyme is phi29.

7. The method of claim 1 wherein the array comprises a plurality of oligonucleotide junction probes, wherein a junction probe is complementary to a junction generated by ligation of the ends of a fragment in the first collection of fragments.

8. The method of claim 7 wherein a plurality of predicted junctions are identified using a computer system and the oligonucleotide probes are designed to detect the presence or absence of a plurality of the predicted junctions.

9. The method of claim 7 wherein the array further comprises a plurality of oligonucleotide probes that are complementary to regions that include a recognition site for said second restriction enzyme.

10. The method of claim 7 wherein the array comprises at least 10,000 different oligonucleotide probe sequences and wherein each probe sequence is present at a different known or determinable location in the array.

11. The method of claim 10 wherein each probe is present on a solid support.

12. The method of claim 11 wherein the solid support is selected from the group consisting of a bead, a plurality of beads, one or more silica chips and one or more glass slides.

13. The method of claim 7 wherein a computer system is used to analyze the hybridization pattern and detect the presence or absence of a plurality of the predicted junctions in a sample and to identify the presence or absence of methylation at a plurality of recognition sites for the second restriction enzyme based on the presence or absence of selected junctions.

14. The method of claim 1 wherein an exonuclease is used to digest linear fragments in the second collection of fragments.

15. The method of claim 14 wherein the exonuclease is Lambda exonuclease.

16. The method of claim 14 wherein the exonuclease is a mixture of Lambda exonuclease and Exonuclease 1.

17. The method of claim 1 wherein the first restriction enzyme is selected from the group consisting of BsaW I, BsoB I, BssS I, Msp I and Taq I.

18. The method of claim 1 wherein the second restriction enzyme is selected from the group consisting of Aat II, Aci I, Acl I, Afe I, Age I, Asc I, Ava I, BmgB I, BsaA I, BsaH I, BspD I, Eag I, Fse I, Fau I, Hpa II, HinP1 I, Nar I, Hin6I, HapII and SnaB I.

19. The method of claim 1 wherein the recognition site of first restriction enzyme and the recognition site of second restriction enzyme differ by at least one base and wherein said second restriction enzyme generates an overhang that is complementary to the overhang generated by the first restriction enzyme.

20. The method of claim 1 wherein the first enzyme is Taq I and the second enzyme is Hpa II.

21. The method of claim 1 wherein the sample is a sample obtained from a source selected from the group consisting of a blood sample, a tissue sample and a tumor sample.

22. The method of claim 1 wherein the sample is a nucleic acid sample obtained from a cell culture.

23. The method of claim 1 further comprising end filling the first collection of fragments to generate blunt ended fragments prior to step (b).

24. The method of claim 1 wherein the step of analyzing the hybridization pattern comprises comparing said hybridization pattern to a second hybridization pattern wherein said second hybridization pattern is obtained by a method comprising:
fragmenting a second sample with said first restriction enzyme to generate a first collection of fragments from said second sample;
adding a ligase to the first collection of fragments from said second sample so that the ends of fragments in the first collection of fragments from said second sample are ligated together to form circular fragments, thereby generating a second collection of fragments from said second sample;
digesting linear fragments in the second collection of fragments from said second sample;
amplifying circular fragments in the second collection of fragments from said second sample to generate a third collection of fragments from said second sample; and,
hybridizing the third collection of fragments from said second sample to an array to obtain said second hybridization pattern.

25. The method of claim 1 wherein the step of analyzing the hybridization pattern comprises comparing said hybridization pattern to a second hybridization pattern wherein said second hybridization pattern is obtained by a method comprising:
fragmenting a second sample with said first restriction enzyme and with a third restriction enzyme that is a methylation insensitive isoschizomer of said second restriction enzyme to generate a first collection of fragments from the second sample;
adding a ligase to the first collection of fragments from the second sample so that the ends of fragments in the first collection of fragments from the second sample are ligated together to form circular fragments, thereby generating a second collection of fragments from the second sample;
odigesting linear fragments in the second collection of fragments from the second sample;
amplifying circular fragments in the second collection of fragments from the second sample to generate a third collection of fragments from the second sample; and,
hybridizing the third collection of fragments from the second sample to an array to obtain the second hybridization pattern.

26. A method of classifying an unknown tumor into a known tumor class comprising:
(a) obtaining a nucleic acid sample from said unknown tumor;
(b) fragmenting the nucleic acid sample with a first restriction enzyme that is insensitive to the methylation status of cytosines and a second restriction enzyme that is sensitive to the methylation status of cytosines to generate a first collection of fragments;
(c) adding a ligase to the first collection of fragments so that the ends of fragments in the first collection of fragments are ligated together to form circular fragments, thereby generating a second collection of fragments;
(d) amplifying circular fragments in the second collection of fragments and optionally digesting linear fragments, to generate a third collection of fragments;
(e) hybridizing the third collection of fragments to an array to obtain a hybridization pattern characteristic of said unknown tumor;
(f) obtaining a plurality of second hybridization patterns characteristic of each of a plurality of known tumor classes, wherein the second hybridization patterns are each generated according to the method of steps (a) to (e);
(g) comparing the first hybridization pattern characteristic of the unknown tumor to each of the second hybridization patterns to identify the second hybridization pattern that most closely matches the first hybridization pattern; and
(h) classifying the unknown tumor in the class of the tumor of known class with the most closely matched second hybridization pattern.

27. The method of claim 26 wherein the array comprises a plurality of oligonucleotide probes that are junction probes, wherein a junction probe is complementary to a junction generated by ligation of the ends of a fragment in the first collection of fragments.

28. The method of claim 27 wherein the junction probes are complementary to junctions present in a computer generated database of junctions predicted to be generated by intramolecular ligation of the ends of fragments in the first collection of fragments.

29. The method of claim 26 wherein the step of amplifying circular fragments comprises rolling circle amplification.

30. The method of claim 26 where the array includes a plurality of junction probes for each junction that vary by at least 1 nucleotide.

31. The method of claim 26 wherein the second collection of fragments is digested with an exonuclease before (d).

32. The method of claim 31 wherein the exonuclease is a mixture of lambda exonuclease and exonuclease I.

33. An array of probes comprising:
at least 100,000 different probes comprising experimental probes and control probes, wherein at least 90% of the probes are experimental probes;
wherein each probe is present at a different, known or determinable, location in the array;
wherein at least 90% of the experimental probes are complementary to genomic target fragments, wherein a plurality of target fragments:
(a) are between 150 and 2000 base pairs when a selected mammalian genome is digested with a first restriction enzyme that recognizes a first recognition site; and
(b) comprise at least one second recognition site for a second restriction enzyme, wherein the second recognition site includes a CpG dinucleotide and said second restriction enzyme does not cleave at the second recognition site when the second recognition site is methylated.

34. The array of claim 33 wherein the second restriction enzyme is HpaII.

35. The array of claim 33 wherein said second restriction enzyme is an isoschizomer of a third restriction enzyme that cleaves at the second recognition site when then second recognition site is methylated.

36. The array of claim 35 wherein said third restriction enzyme is MspI.

37. The array of claim 33 wherein said selected mammalian genome is selected from the group consisting of the human genome, the mouse genome and the rat genome.

38. A method of determining the presence or absence of methylation at a plurality of cytosines in a nucleic acid sample, comprising:
(a) digesting the nucleic acid sample with a first restriction enzyme with a recognition site that includes a cytosine, wherein said first restriction enzyme is methylation sensitive;
(b) adding a ligase to the fragments generated in step (a) to generate circular fragments;
(c) digesting the products of step (b) with a restriction enzyme that is methylation dependent;
(d) digesting the products of step (c) to remove single stranded fragments;
(e) amplifying the products of step (d) using an amplification method that amplifies circular fragments;
(f) fragmenting and labeling the products of step (e);
(g) hybridizing the products of step (f) to an array of probes and detecting a resulting hybridization pattern; and
(h) analyzing the hybridization pattern to determine the methylation state of a plurality of cytosines in the nucleic acid sample.

39. The method of claim 38 wherein the enzyme that is methylation dependent is McrBC.

40. The method of claim 38 wherein the amplification method is rolling circle amplification.

41. The method of claim 38 wherein the products of step (e) are fragmented by treatment with DNase.

42. The method of claim 38 wherein dUTP is included during in step (e) and the products of step (e) are fragmented by incubation with uracil DNA glycosidase and an AP endonuclease.

43. The method of claim 42 wherein the AP endonuclease is selected from the group consisting of Endo IV and Ape I.

44. The method of claim 38 wherein the fragments generated in step (f) are end labeled by TdT and DLR.

45. The method of claim 38 wherein the array comprises a plurality of probes complementary to a plurality of genomic regions.

46. The method of claim 38 wherein step (h) comprises: (i) using a computer to predict fragments resulting from step (a) by in silico digestion; (ii) identifying a plurality of fragments from step (i) that include at least one recognition site for the methylation dependent enzyme; (iii) identifying fragments in the plurality identified in step (ii) that are present in the products of step (f).

47. A method of analyzing the methylation state of at least one cytosine in a nucleic acid sample, comprising:
(a) fragmenting the nucleic acid sample to obtain a plurality of linear fragments;
(b) ligating a plurality of the fragments to form circular fragments;
(c) enriching the product of step (b) for methylated circular fragments by a method comprising: mixing the circular fragments with one or more methylation sensitive restriction enzymes and an exonuclease;
(d) amplifying circular fragments from step (c) to generate an amplification product;
(e) fragmenting and labeling the amplification product;
(f) hybridizing the amplification product to an array of nucleic acid probes to obtain a hybridization pattern; and,
(g) analyzing the hybridization pattern to determine the methylation status of at least one cytosine.

48. The method of claim 47 wherein the sample is obtained from a source selected from the group consisting of a tumor, a cell culture, a normal tissue, saliva, skin cells and blood.

49. The method of claim 47 wherein the array comprises a plurality of junction probes.

50. The method of claim 47 wherein the array comprises a plurality of tiled probes.

51. A method of obtaining a sample that is enriched for methylated sequences from a first sample, comprising:
obtaining said first sample, wherein said first sample comprises methylated and unmethylated genomic DNA;
fragmenting said first sample using a restriction enzyme that is methylation insensitive to obtain a fragmented sample;
treating said fragmented sample with a ligase to circularize at least some of the fragments in the fragmented sample to generate a circularized sample;
treating said circularized sample with at least one methylation sensitive restriction enzyme to obtain a sample that is enriched for methylated sequences.

52. The method of claim 51 wherein the recognition site for the methylation insensitive enzyme does not include a cytosine.

53. The method of claim 51 wherein the recognition site for the methylation insensitive enzyme does include a cytosine.

54. The method of claim 51 wherein said first sample is obtained from a source selected from the group consisting of a tumor, a cell culture, a normal tissue, saliva, skin cells and blood.

55. A method of obtaining a sample that is enriched for unmethylated sequences from a first sample, comprising:
obtaining said first sample, wherein said first sample comprises methylated and unmethylated genomic DNA;
fragmenting said first sample using a restriction enzyme that is methylation sensitive to obtain a fragmented sample;
circularizing at least some of the fragments in the fragmented sample to generate circular fragments;
generating linear fragments from circular fragments that contain a methylated recognition site for the methylation dependent restriction enzyme;
digesting a plurality of said linear fragments; and,
amplifying circular fragments to obtain a sample that is enriched for unmethylated sequences.

56. The method of claim 55 wherein the methylation dependent restriction enzyme is McrBC.

57. The method of claim 55 wherein the linear fragments are digested with an exonuclease.

58. The method of claim 57 wherein the exonuclease is Lambda exonuclease.

59. The method of claim 57 wherein the exonuclease is a mixture of Lambda exonuclease and Exonuclease I.

60. The method of claim 55 wherein the step of amplifying circular fragments is by a method comprising rolling circle amplification with a strand displacing DNA polymerase.

61. The method of claim 60 wherein the strand displacing DNA polymerase is phi 29 DNA polymerase or Bst DNA polymerase.

62. The method of claim 57 wherein the sample is obtained from a source selected from the group consisting of a tumor, a cell culture, a normal tissue, saliva, skin cells and blood.

63. A method for determining the methylation status of a plurality of cytosines in a sample comprising genomic DNA, said method comprising:
(a) fragmenting the sample with a first restriction enzyme that is insensitive to the methylation status of cytosines and a second restriction enzyme that is sensitive to the methylation status of cytosines to generate a first collection of fragments;
(b) adding a ligase to the first collection of fragments so that the ends of fragments in the first collection of fragments are ligated together to form circular fragments, thereby generating a second collection of fragments;
(c) hybridizing a plurality of molecular inversion probes to the third collection of fragments wherein the molecular inversion probes include a 5′ region that is complementary to the first half of a predicted junction and a 3′ region that is complementary to the second half of a predicted junction so that when the predicted junction is present the molecular inversion probe can hybridize to the junction so that the 5′ and 3′ ends of the molecular inversion probe can be ligated together;
(d) ligating the ends of a plurality of the molecular inversion probes; and
(e) removing linear molecular inversion probes;
(f) amplifying the molecular inversion probes remaining after step (e); and
(g) detecting the presence of a plurality of molecular inversion probe amplicons in the amplified product of step (f), wherein the presence of a molecular inversion probe amplicon is indicative of the presence of a predicted junction; and
(h) determining the methylation status of selected cytosines.

64. The method of claim 63 wherein each molecular inversion probe comprises a tag sequence that is different from the tag sequence in every other molecular inversion probe in the plurality.

65. The method of claim 64 wherein step (g) includes the step of hybridizing at least a portion of the molecular inversion probe amplicons to an array of probes that are complementary to the tag sequences.

66. The method of claim 65 wherein the molecular inversion probes comprise a first universal priming site and a second universal priming site and the molecular inversion probes are cleaved at a position between said first and second universal priming sites after circularization and amplified by PCR using primer complementary to said first and second universal priming sites.

67. A computer implemented method for determining the methylation status of a plurality of cytosines in a sample comprising:
providing a plurality of signals where in each signal represents the level of a junction in a sample;
providing a database of junction sequences;
comparing the plurality of signals to the database of junction sequences to identify a plurality of sequences that are present in the sample at a level above background; and,
analyzing the pattern of junctions to determine the methylation status of a plurality of cytosines.

68. The method of claim 67 wherein the database of junction sequences is provided by computer implemented modeling enzymatic digestion of known genomic sequence with a methylation sensitive enzyme in the presence or absence of methylation at a plurality of recognition sites for the methylation sensitive enzyme.

69. A computer software product comprising:
computer program code that inputs a plurality of signals where each of said signals reflects the relative level of a junction in a sample, wherein junctions are formed by intramolecular ligation of restriction fragments;
computer program code that compares the relative level of a plurality of junctions to a database of junctions, wherein the formation of at least some of said junctions is dependent on the presence of methylation at a restriction site; and,
a computer readable media storing said computer program codes.

70. The computer software product of claim 69 wherein said database comprises more than 10,000 junctions and wherein the formation of more than 5,000 of said junctions is dependent on the methylation of a restriction site.

71. A method of analyzing the methylation state of at least one cytosine in a nucleic acid sample, comprising:
(a) fragmenting the nucleic acid sample to obtain a plurality of linear fragments;
(b) ligating a plurality of the fragments to form circular fragments;
(c) enriching the product of step (b) for unmethylated circular fragments by a method comprising: mixing the circular fragments with one or more methylation dependent restriction enzymes and an exonuclease;
(d) amplifying circular fragments from step (c) to generate an amplification product;
(e) fragmenting and labeling the amplification product;
(f) hybridizing the amplification product to an array of nucleic acid probes to obtain a hybridization pattern; and,
(g) analyzing the hybridization pattern to determine the methylation status of at least one cytosine.

72. The method of claim 71 where the methylation sensitive enzyme is McrBC.
